(19) United States
(12) Patent Application Publication
(10) Pub. No.: US 2025/0026779 A1
(43) Pub. Date: Jan. 23, 2025

(54) ETHYL MODIFIED RNA CAPS AND METHODS OF USE

(71) Applicant: NUTCRACKER THERAPEUTICS, INC., Emeryville, CA (US)

(72) Inventors: Colin James McKinlay, Fremont, CA (US); Wan Lu Woo-Derleth, Richmond, CA (US)

(21) Appl. No.: 18/759,011

(22) Filed: Jun. 28, 2024

Related U.S. Application Data
(60) Provisional application No. 63/524,050, filed on Jun. 29, 2023.

(51) Int. Cl. C07H 21/02; A61K 31/7105; C12P 19/34

(52) U.S. Cl. CPC C07H 21/02; A61K 31/7105; C12P 19/34

(57) ABSTRACT

Provided herein are compounds that are cap analogs for polynucleotides, e.g., RNA molecules, such as mRNA molecules. Also provided are capped polynucleotides, e.g., capped RNA molecules, such as capped mRNA molecules, wherein the 5′ end of the RNA molecule comprises a cap analog disclosed herein, drug products comprising the capped RNA molecules, methods for making capped polynucleotides disclosed herein, and kits for making the capped polynucleotides.

### BACKGROUND

The present disclosure generally relates to RNA cap analogs, polynucleotides (e.g., mRNAs) containing the cap analogs, and methods of making and using the cap analogs.

The naturally-occurring eukaryotic mRNA has a cap structure comprising an N7-methylated guanosine (m7G or 7mG) linked to the first nucleotide of the mRNA via a reverse 5′ to 5′ triphosphate linkage (5′ ppp). The mRNA cap has an essential role in cap-dependent initiation of protein synthesis and functions as a protective group from 5′ to 3′ exonuclease cleavage and a unique identifier for recruiting protein factors for pre-mRNA splicing, polyadenylation and nuclear export. It also acts as the anchor for the recruitment of initiation factors that initiate protein synthesis and the 5′ to 3′ looping of mRNA during translation.

Capping can be achieved on synthetically-derived mRNA using multiple strategies including post-transcriptional enzymatic capping and co-transcriptional capping. Several enzymatic steps are required for the conversion of the 5-triphosphorylated end of an mRNA transcript into a capped structure. The enzymes involved in these steps are referred to as “capping enzymes.” Co-transcriptional capping uses a “capping reagent” which can be incorporated into a growing mRNA chain during the transcriptional process without the need for additional enzymatic steps. The RNAs that result from these enzymatic steps are referred to as “5′ capped RNAs” or simply “capped RNAs.”

The mRNA cap structure is implicated in many aspects of mRNA efficacy, including translation efficacy, mRNA stability, and mRNA immunogenicity. However, little is known about the medicinal chemistry of how these cap structures bind to the relevant enzymes in either the capping process (i.e. during mRNA synthesis), or to the translation machinery enzymes such as eIF4E.

There remains a need for synthetic cap analogs that efficiently cap polynucleotides and provide polynucleotides with desired biological properties, such as capped RNAs with desirable translation efficiency.

### SUMMARY

Provided herein are compounds of Formula (I), or a stereoisomer, tautomer or pharmaceutically acceptable salt thereof:

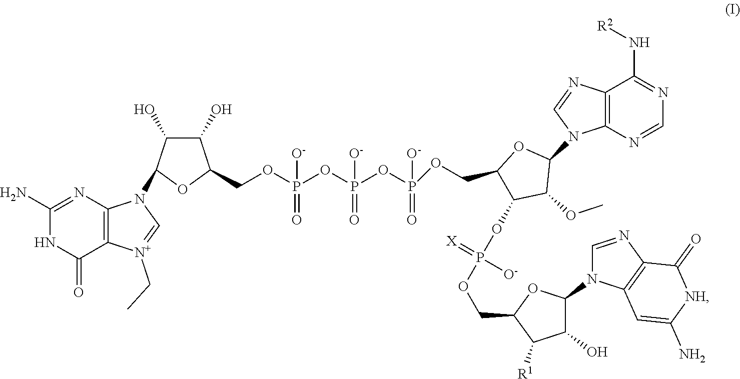

wherein X is O or S, R¹ is OH or a polynucleotide, and R² is H or Me.

Also provided are RNA molecules, wherein the 5′ end of the RNA molecule comprises a compound, stereoisomer, tautomer, or salt disclosed herein. Further provided are drug products comprising a capped RNA molecule disclosed herein and one or more pharmaceutically acceptable excipients. Further provided are kits for capping an RNA molecule comprising a compound disclosed herein, or stereoisomer, tautomer, or salt thereof, and an RNA polymerase. Further provided are methods for making a capped RNA molecule from a polynucleotide template by in vitro transcription, comprising: (a) combining a plurality of nucleotides, the polynucleotide template and an RNA polymerase to produce a reaction mix; (b) incubating the reaction mix; and (c) adding a compound disclosed herein, or a stereoisomer, tautomer, or salt thereof to the mix in order to produce the capped RNA molecule.

### BRIEF DESCRIPTION OF THE DRAWINGS

FIG. 1A shows the “IVT”, or in vitro transcription yield for transcription of mRNAs containing RNA cap structures Cap 1, Compound 1, or Compound 2.

FIG. 1B shows the in vitro expression of mRNAs containing either RNA cap structure Cap 1 or RNA cap structure Compound 1.

FIG. 1C shows the in vivo expression of mRNAs containing either RNA cap structure Cap 1 or RNA cap structure Compound 1.

FIG. 1D shows the levels of dsRNA produced during IVT reactions with mRNAs containing either RNA cap structure Cap 1 or RNA cap structure Compound 1, as determined by sandwich ELISA.

FIG. 2A shows the “IVT”, or in vitro transcription yield for transcription of mRNAs containing RNA cap structures Cap 1, Cap 3, Cap 3, Compound 3, or Compound 4.

FIG. 2B shows the in vitro expression of mRNAs containing RNA cap structures Cap 1, Cap 3, Cap 3, Compound 3, or Compound 4.

FIG. 2C shows the in vivo expression of mRNAs containing RNA cap structures Cap 1, Cap 3, Cap 3, or Compound 3.

FIG. 2D shows the levels of dsRNA produced during IVT reactions with mRNAs containing RNA cap structures Cap 1, Cap 3, Cap 3, or Compound 3, as determined by sandwich ELISA.

FIG. 3A shows the expression of mRNAs containing no cap structures, or RNA cap structures Cap 1 or Compound 3.

FIG. 3B shows the degree of IFNγ stimulation by mRNAs containing no cap structures, or RNA cap structures Cap 1 or Compound 3.

FIG. 3C shows the degree of IFNα stimulation by mRNAs containing no cap structures, or RNA cap structures Cap 1 or Compound 3.

FIG. 3D shows the degree of IP-10 stimulation by mRNAs containing no cap structures, or RNA cap structures Cap 1 or Compound 3.

FIG. 3E shows the degree of IL-1a stimulation by mRNAs containing no cap structures, or RNA cap structures Cap 1 or Compound 3.

FIG. 3F shows the degree of TNFα stimulation by mRNAs containing no cap structures, or RNA cap structures Cap 1 or Compound 3.

### DETAILED DESCRIPTION

Provided herein are compounds that are cap analogs for polynucleotides, e.g., RNA molecules, such as mRNA molecules. Also provided are capped polynucleotides, e.g., capped RNA molecules, such as capped mRNA molecules, wherein the 5′ end of the RNA molecule comprises a cap analog disclosed herein, and drug products comprising the capped RNA molecules. Also provided are methods for making capped polynucleotides disclosed herein, and kits for making the capped polynucleotides.

Capped polynucleotides, such as mRNAs, can be prepared based on in vitro transcription (IVT) of a DNA template by post-transcriptional capping or co-transcriptional capping. “In vitro transcription” (IVT), as used herein, refers to a cell-free reaction in which a DNA template is copied by an RNA polymerase to produce RNA molecules that are encoded by the DNA template.

In post-transcriptional capping, the RNA from IVT is capped via a series of enzymatic capping reactions. In co-transcriptional capping, cap analogues are added directly to the IVT. Synthetic cap analogs have a potential for increased capping efficiency, decreased de-capping and reverse capping. The RNAs capped with these analogs have reduced degradation, increased nuclease resistance, increased translation yield, and reduced immunogenicity.

### Cap Analogs and Capped Polynucleotides

Provided herein are compounds that are synthetic cap analogs and methods of making capped polynucleotides, such as mRNAs, with the cap analogs.

The cap analog as disclosed herein is a compound of Formula (I), or a stereoisomer, tautomer or pharmaceutically acceptable salt thereof:

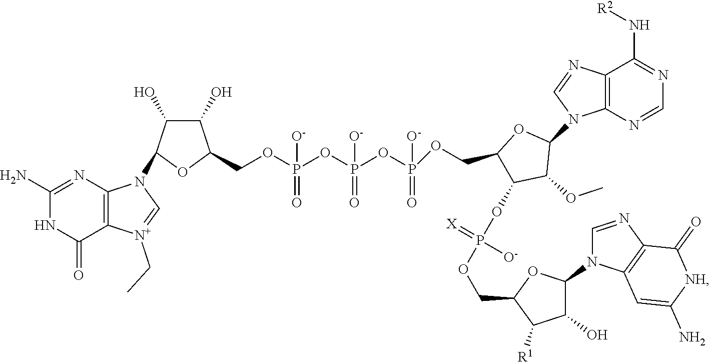

wherein X is O or S, R¹ is OH or a polynucleotide, and R² is H or Me. In some cases, the stereochemistry of the phosphorus having the X substitution is (R) (i.e., the compound is an R-P compound of Formula (I)). In some cases, the stereochemistry is (S) (i.e., the compound is an S-P compound of Formula (I)). In some cases, X is O. In some cases, X is S. In some cases, R¹ is OH. In various cases, R¹ is a polynucleotide. In various cases, R² is H. In various cases, R² is Me.

Specific compounds of Formula (I) include compounds listed in Table 1, below.

### Nucleobases, Nucleosides, and Nucleotides

In some instances, the cap analogs as described herein are attached to polynucleotides (i.e., R¹ is a polynucleotide) that comprise at least one non-naturally occurring nucleobase and/or at least one modified nucleobase. The terms “modified” or, as appropriate, “modification” refer to structural and/or chemical modifications with respect to A, G, U, or C nucleobases, nucleosides, and/or nucleotides. Nucleotides in the cap analogs of the present disclosure may comprise non-standard nucleotides, such as non-naturally occurring nucleotides or chemically synthesized nucleotides. The cap analogs of the present disclosure may include any useful modification, such as to the sugar, the nucleobase, or the internucleoside linkage (e.g. to a linking phosphate, to a phosphodiester linkage, or to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain cases, modifications (e.g., one or more modifications) are present in each of the sugar and the internucleoside linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof.

Different sugar modifications, nucleotide modifications, and/or internucleoside linkages (e.g., backbone structures) may exist at various positions in a cap analog attached to a polynucleotide. For example, the sugars in the nucleotides may be interpedently, for each position, selected from ribose and deoxyribose, and may comprise modifications such as but not limited to 2′-O-alkyl, 2′-O-methoxyethyl, 2′-O-allyl, 2′-O-alkylamine, 2′-fluororibose 2′-deoxyribase, and locked nucleic acid (LNA). The bases in the nucleotides may be independently, for each position, selected from adenine, uridine, guanine, or cytidine or analogs of adenine, uridine, guanine, or cytidine, such as modified adenine, uridine, guanine, or cytidine. Non-limiting examples of adenine, uridine, guanine, and cytidine analogs and modified adenine, uridine, guanine, and cytidine include N6-methyladenine, N1-methylademine, N6-2′-O-dimehtyladenosine, pseudouridine, N1-methypseudouridine, 5-iodouridine, 4-thiouridine, 2-thiouridine, 5-methyluridine, pseudoisocytosine, 5-methoxycytosine, 2-thiocytosine, 5-hydroxycytosine, N4-methylcytosine, 5-hydroxymethylcytosine, hypoxanthine, N1-methylguanine, 06-methylguanine, 1-methyl-guanosine, N2-methyl-guanosine, N2,N2-dimethyl-guanonsine, 2-methyl-guanosine, N7-methyl-guanosine, 1-methyl-guanosine, N2,N7-dimethyl-guanosine, and isoguanine.

In some instances, the modifications include bicyclic derivatives of the nucleotides (LNA, ENA, CLNA, CENA, AENA etc.), acyclic nucleotides (UNA, PNA, etc.) or nucleotides containing pyranose ring (ANA, HNA) instead of ribose.

In some instances, the modification may be on the backbone. Non-limiting examples include the replacement of phosphate group (PO) with phosphorothioate (PS) or boranophosphonate (PB) groups, the replacement of the 3′,5′-phosphodiester bond with 2′,5′-bond or the amide bond instead of the ester bond, etc.

In some embodiments, the modification may be on the nucleobases. For example, uridine (U) may be replaced with pseudouridine (ψ), 2-thiouridine (s2U), dihydrouridine (D), 5-bromo-U, 5-iodo-U, etc. A purine may be replaced with a 2,6-diaminopurine.

The polynucleotides described herein can include various substitutions and/or insertions from native or naturally occurring polynucleotides, e.g., in addition to the modification on the 5′ terminal mRNA cap moieties disclosed herein. As used herein, when referring to a polynucleotide, the terms “chemical modification” or, as appropriate, “chemically modified” refer to modification with respect to adenosine (A), guanosine (G), uridine (U), thymidine (T) or cytidine (C) ribo- or deoxyribnucleosides and the internucleoside linkages in one or more of their position, pattern, percent or population. Generally, herein, these terms are not intended to refer to the ribonucleotide modifications in naturally occurring 5′-terminal mRNA cap moieties.

The modifications may be various distinct modifications. In some embodiments, the regions may contain one, two, or more (optionally different) nucleoside or nucleotide modifications. In some embodiments, a modified polynucleotide introduced to a cell may exhibit reduced degradation in the cell as compared to an unmodified polynucleotide.

Modifications of the polynucleotides of the disclosure include, but are not limited to, those listed in detail below. The polynucleotide may comprise modifications which are naturally occurring, non-naturally occurring or the polynucleotide can comprise both naturally and non-naturally occurring modifications.

The polynucleotides of the disclosure can include any modification, such as to the sugar, the nucleobase, or the internucleoside linkage (e.g., to a linking phosphate, to a phosphodiester linkage, or to the phosphodiester backbone). One or more atoms of a pyrimidine or purine nucleobase may be replaced or substituted with optionally substituted amino, optionally substituted thiol, optionally substituted alkyl (e.g., methyl or ethyl), or halo (e.g., chloro or fluoro).

In certain embodiments, modifications (e.g., one or more modifications) are present in each of the sugar and the internucleoside linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. Additional modifications are described herein.

Non-natural modified nucleotides may be introduced to polynucleotides during synthesis or post-synthesis of the chains to achieve desired functions or properties. The modifications may be on internucleotide linkage, the purine or pyrimidine bases, or sugar. The modification may be introduced at the terminal of a chain or anywhere else in the chain; with chemical synthesis or with a polymerase enzyme. Any of the regions of the polynucleotides may be chemically modified.

The present disclosure provides for polynucleotides comprising unmodified or modified nucleosides and nucleotides and combinations thereof. As described herein “nucleoside” is defined as a moiety containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphodiester linkages, in which case the polynucleotides would comprise regions of nucleotides. Any combination of base/sugar or linker may be incorporated into the polynucleotides of the disclosure.

Modifications of polynucleotides (e.g., RNA polynucleotides, such as mRNA polynucleotides), including but not limited to chemical modification, that are useful in the compositions, methods and synthetic processes of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 21-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-Iuoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 21-O-methylcytidine; 5,2′-O-dimethylcytidine; 5-formy1-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; a-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,21-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-I-deaza-pseudoisocytidine; 1-methyl-pseudoisocytidine; 2-methoxy-5-methyl-cytidine; 2-methoxy-cytidine; 2-thio-5-methyl-cytidine; 4-methoxy-I-methyl-pseudoisocytidine; 4-methoxy-pseudoisocytidine; 4-thio-I-methyl-1-deaza-pseudoisocytidine; 4-thio-I-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 21-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-0-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; a-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-ethyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-PentenylaminomethyOuridine TP; 5-propynyl uracil; a-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-I-methyl-1-deaza-pseudouridine; 2-thio-I-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-I-methyl-pseudouridine; 4-thio-pseudouridine; 5-aza-uridine; Dihydropseudouridine; (±)1-(2-Hydroxy propyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bi s-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxy butyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxy benzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxy benzyl)pseudouridineTP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy-ethoxy)-propionyl] pseudouridine TP; 1-13-[2-(2-Amino ethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-rifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-AzidouridineTP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-12-(2-[2-(2-ethoxy)-ethoxy]-ethoxy)-ethoxy1]propionic acid; Pseudouridine TP 1-[3-{24242-12(2-ethoxy)-ethoxy 1-ethoxy]-ethoxy)-ethoxy1]propionic acid; Pseudouridine TP 1-[3-12-(2-[2-ethoxy]-ethoxy)-ethoxyllpropionic acid; Pseudouridine TP 143-12-(2-ethoxy)-ethoxyll propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-I-yl: 1,3-(diaza)-2-(oxo)-phenthiazin-I-y1; 1,3-(diaza)-2-(oxo)-phenoxazin-I-y1; 1,3,5-(triaza)-2,6-(dioxa)-naphthalene; 2 (amino)purine; 2,4,5-(trimethyl)pheny1; 2′ methyl, 2′amino, 2′azido, 2′fluro-cytidine; 21 methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-y1; 7-(aminoalkylhydroxy)-1-(aza)-2-(thio)-3-(aza)-phenthiazin-I-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-I-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-I-y1; 7-(guanidiniumalkylhydroxy)-1-(aza)-2-(thio)-3-(aza)-phenoxazin-I-y1; 7-(guanidiniumalkylhydroxy)-1,3-(di aza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-y1; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-y1; para-substituted-6-phenyl-pyrrolo-pyrimidin-2-on-3-y1; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidineTP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 21-O-methyl uridine, 1-methyl-pseudouridine (mli), 1-ethyl-pseudouridine (e1y), 5-methoxy-uridine (mo5U), 5-methyl-cytidine (m5C), a-thio-guanosine, a-thio-adenosine, 5-cyano uridine, 4′-thio uridine 7-deaza-adenine, 1-methyl-adenosine (ml A), 2-methyl-adenine (m2A), N6-methyl-adenosine (m6A), and 2,6-Diaminopurine, (I), 1-methyl-inosine (mi1), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (ml G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethy1-2-thiouridine, 5-carboxymethylaminomethy1-2-geranylthiouridine, 5-carboxymethylaminomethy1-2-selenouridine, 5-cyanomethyluridine, 5-hydroxycytidine, 5-methylaminomethy1-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQObase, preQ1base, and two or more combinations thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polyribonucleotide (e.g., RNA polyribonucleotide, such as mRNA polyribonucleotide) includes a combination of at least two (e.g., 2, 3, 4 or more) of the aforementioned modified nucleobases. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m1ψ), 1-ethyl-pseudouridine (e1ψ), 5-methoxy-uridine (mo5U), 5-methyl-cytidine (m5C), pseudouridine (ψ), a-thio-guanosine and a-thio-adenosine. In some embodiments, the polyribonucleotide includes a combination of at least two (e.g., 2, 3, 4 or more) of the aforementioned modified nucleobases, including but not limited to chemical modifications.

In some embodiments, polynucleotides (e.g., RNA polynucleotides, such as mRNA polynucleotides) comprise pseudouridine (ψ) and 5-methyl-cytidine (m5C). In some embodiments, the polyribonucleotides (e.g., RNA, such as mRNA) comprise 1-methyl-pseudouridine (m1ψ). In some embodiments, the polyribonucleotides (e.g., RNA, such as mRNA) comprise 1-ethyl-pseudouridine (e1ψ). In some embodiments, the polyribonucleotides (e.g., RNA, such as mRNA) comprise 1-methyl-pseudouridine (m1w) and 5-methyl-cytidine (m5C). In some embodiments, the polyribonucleotides (e.g., RNA, such as mRNA) comprise 1-ethyl-pseudouridine (e1ψ) and 5-methyl-cytidine (m5C). In some embodiments, the polyribonucleotides (e.g., RNA, such as mRNA) comprise 2-thiouridine (s2U). In some embodiments, the polyribonucleotides (e.g., RNA, such as mRNA) comprise 2-thiouridine and 5-methyl-cytidine (m5C). In some embodiments, the polyribonucleotides (e.g., RNA, such as mRNA) comprise methoxy-uridine (mo5U). In some embodiments, the polyribonucleotides (e.g., RNA, such as mRNA) comprise 5-methoxy-uridine (mo5U) and 5-methyl-cytidine (m5C). In some embodiments, the polyribonucleotides (e.g., RNA, such as mRNA) comprise 21-O-methyl uridine. In some embodiments, the polyribonucleotides (e.g., RNA, such as mRNA) comprise 21-O-methyl uridine and 5-methyl-cytidine (m5C). In some embodiments, the polyribonucleotides (e.g., RNA, such as mRNA) comprise N6-methyl-adenosine (m6A). In some embodiments, the polyribonucleotides (e.g., RNA, such as mRNA) comprise N6-methyl-adenosine (m6A) and 5-methyl-cytidine (m5C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 1-methyl-pseudouridine, meaning that all uridine residues in the mRNA sequence are replaced with 1-methyl-pseudouridine. Similarly, a polynucleotide can be uniformly modified for any type of nucleoside residue present in the sequence by replacement with a modified residue such as those set forth above.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G-HU, G-FC, U+C, A+G-HU, A+G-FC, G-HU+C or A+G+C.

In some embodiments, the polynucleotide contains 1% to 100% modified nucleotides (either in relation to overall nucleotide content, or in relation to one or more types of nucleotide, i.e., any one or more of A, G, U or C) or any intervening percentage (e.g., 1% to 5%, 1% to 10%, 1% to 20%, 1% to 25%, 1% to 50%, 1% to 60%, 1% to 70%, 1% to 80%, 1% to 90%, 1% to 95%, 10% to 20%, 10% to 25%, 10% to 50%, 10% to 60%, 10% to 70%, 10% to 80%, 10% to 90%, 10% to 95%, 10% to 100%, 20% to 25%, 20% to 50%, 20% to 60%, 20% to 70%, 20% to 80%, 20% to 90%, 20% to 95%, 20% to 100%, 50% to 60%, 50% to 70%, 50% to 80%, 50% to 90%, 50% to 95%, 50% to 100%, 70% to 80%, 70% to 90%, 70% to 95%, 70% to 100%, 80% to 90%, 80% to 95%, 80% to 100%, 90% to 95%, 90% to 100%, and 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA molecules of the disclosure comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethyl-2-thio-uridine(Tm5s2U), 1-taurinomethyl-4-thio-pseudouridine, 5-methyl-uridine (m5U, i.e., having the nucleobase deoxythymine), 1-methyl-pseudouridine (m1ψ), 1-ethyl-pseudouridine (e1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 kv), 5-(isopentenylaminomethyl)uridine (inm5U), 5-(isopentenylaminomethyl)-2-thio-uridine (inm5s2U), a-thio-uridine, 2′-O-methyl-uridine (Urn), 5,2′-O-dimethyl-uridine (m5Um), 2′-O-methyl-pseudouridine (ψm), 2-thio-2′-O-methyl-uridine (s2Um), 5-methoxycarbonylmethy1-2′-O-methyl-uridine (mcm5Um), 5-carbamoylmethyl-2′-O-methyl-uridine (ncm5Um), 5-carboxymethylaminomethy1-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine(ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (miAm), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQi), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine N2-methyl-guanosine (m2G), N2,N2-dimethyl-guanosine (m22G), N2,7-dimethyl-guanosine (m2′7G), N2, N2,7-dimethyl-guanosine 8-oxo-guanosine, 7-methyl-8-oxo-guanosine, 1-methyl-6-thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (miGm), N2,7-dimethyl-2′-O-methyl-guanosine (m2′7Gm), 2′-O-methyl-inosine (Im), 1,2′-O-dimethyl-inosine 2′-O-ribosylguanosine (phosphate) (Gr(p)), 1-thio-guanosine, 06-methyl-guanosine, 2′-F-ara-guanosine, and 2′-F-guanosine.

### Methods of Making the Cap Analogs

The cap analogs of the present disclosure can be prepared with various synthetic strategies known in the art using general chemical synthetic principles and techniques. In a rational approach, the cap analogs are constructed from their structural components. These components can be covalently bonded to one another through functional groups, as is known in the art, where such functional groups may be present on the components or introduced onto the components using one or more steps. Functional groups that may be used in covalently bonding the components together to produce the cap analogs include but not limited to hydroxy, sulfhydryl, or amino groups. The particular portions of the different components that are modified to provide for covalent linkage are chosen so as not to substantially adversely interfere with other portions of the components. When necessary and/or desired, certain moieties on the components may be protected using blocking groups, as is known in the art, see, e.g., Green & Wuts, Protective Groups in Organic Synthesis (John Wiley & Sons) (1991).

It is to be understood that the particular process conditions for preparing the cap analogs described herein may be adjusted or selected accordingly to provide the desired physical properties. In some instances, the cap analogs are prepared with methods according to “Chemical synthesis and characterization of 7-methylguanosine cap analogues,” by E. Darzynkiewicz et al., Biochem., vol. 24, pp. 1701-1707 (1985).

### Methods of Using the Cap Analogs to Prepare Polynucleotides

The cap analogs of the present disclosure can be used for capping an RNA, such as in an in vitro transcription (IVT) reaction, co-transcriptionally.

In some instances, the method of capping an RNA comprise:
combining NTPs, a polynucleotide template, a cap analog and an RNA polymerase to produce a reaction mix, and
incubating the reaction mix under conditions suitable for IVT of the polynucleotide template to produce a capped RNA copy of the polynucleotide template.

In some instances, the transcription yield from the polynucleotide template to the capped RNA is greater than about 75%, such as about 80%, about 85%, about 90% or about 95%.

In some instances, the capping efficiency is at least about 90%, such as about 90%, about 91%, about 92%, about 93%, about 94%, about 95%, about 96%, about 97%, about 98%, about 99%. The capped RNA produced from this method can potentially be used without any post-transcriptional enzymatic capping reactions.

The capping efficiency can be measured with any known method in the art. As a non-limiting example, mass spectrometry (MS), Liquid chromatography mass spectrometry (LC/MS), and/or liquid chromatography such as high-performance liquid chromatography (HPLC) can be utilized to measure capped and uncapped RNA and calculate capping efficiency. In some other non-limiting examples, a detectable label may be attached to the cap analog in order to measure capping efficiency.

The capped RNA produced with this method have higher stability, are more resistant to nucleases, have the same or less immunogenicity, and/or have the same or higher translation efficiency. In some instances, the half-life of the capped RNA is more than that of a corresponding natural RNA molecule in a cellular environment.

NTPs (i.e., GTP, CTP, UTP and ATP), as used herein, include naturally occurring or modified NTPs.

In some instances, the polynucleotide template is a DNA template. The DNA template can comprise any desired sequence that encodes naturally occurring or modified mRNA, tRNA, guide RNA, small inhibiting RNA (siRNA), small activating RNA (saRNA), or a microRNA. The DNA may be double-stranded.

The reaction mixture may be incubated at a temperature of between about 30° C. to about 60° C., such as about 37° C., between about 30° C. to about 40° C., between about 40° C. to about 50° C., or between about 50° C. to about 60° C. The reaction mixture may be incubated for a period of at least 30 min, such as about 40 min, about 50 min, about 60 min, about 1.5 hr, about 2 hr, about 2.5 hr, about 3 hr, about 4 hr, about 5 hr, about 6 hr, about 7 hr, about 8 hr, about 9 hr, about 10 hr, about 11 hr, about 12 hr, or more.

The RNA polymerase may be any known RNA polymerase, including natural ones and synthetic ones. In some instances, the RNA polymerase may be thermostable.

In some instances, the capped RNA can be prepared with solid phase synthesis. Solid-phase synthesis is carried out on a solid support held between filters, in columns that enable all reagents and solvents to pass through freely. Coupling agents, protection groups and cleaving agents can be selected according to methods known to a person in the art.

According to certain embodiments of the disclosure, the cap analogs for use herein comprise an RNA comprising at least one region encoding a peptide (e.g., a polypeptide), or protein, or functional fragment of the foregoing. As used herein, “functional fragment” refers to a fragment of a peptide, (e.g., a polypeptide), or protein that retains the ability to induce an immune response. In one embodiment, the coding RNA is selected from the group consisting of mRNA, viral RNA, retroviral RNA, and self-replicating RNA. In some embodiments, the RNA encodes a viral peptide (e.g., a viral polypeptide), a viral protein, or functional fragment of the foregoing. In various cases, the RNA encodes for a human papillomavirus (HPV) protein, a variant thereof, or a functional fragment of any of the foregoing. In some cases, the RNA encodes for a HPV E6 protein (or a variant thereof), a HPV E7 protein (or a variant thereof), a combination thereof, or a functional fragment of any of the foregoing. In some cases, the HPV protein is from HPV subtype HPV 16, 18, 31, 33, 35, 39, 45, 51, 52, 56, 58, 59, 66, and/or 68. In various cases, the HPV protein is from HPV subtype HPV 16 and/or 18. In some cases, the RNA encodes for a viral spike protein or a functional fragment thereof. In some cases, the RNA encodes for a SARS-Related coronaviruses (e.g., severe acute respiratory syndrome coronavirus-2, (SARS-CoV-2), severe acute respiratory syndrome coronavirus (SARS-CoV), Middle East respiratory syndrome coronavirus (MERS-CoV), human coronavirus 229E (HCoV-229E), human coronavirus 0C43 (HCoV-0C43), human coronavirus HKU1 (HCoV-HKU1), and/or human coronavirus NL63 (HCoV-NL63)). In various embodiments, the RNA encodes for a SARS-CoV spike (S) protein, a variant thereof, or a functional fragment any of the foregoing. In some cases, the RNA encodes for an influenza protein, a variant thereof, or a functional fragment of any of the foregoing. In various embodiments, the RNA encodes for influenza hemagglutinin (HA), or a functional fragment thereof. In some embodiments the influenza A virus, has HA of a subtype selected from the group consisting of H1, H2, H3, H4, H5, H6, H7, H8, H9, H10, H11, H12, H13, H14, H15, and H16. In various embodiments, the influenza subtype is HA strain H1, H2, H3 or H5. In some embodiments, the RNA encodes for a combination of the foregoing.

Non-limiting examples of contemplated viruses for which the cap analogs can encode, include: Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simplex, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simplex, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In some embodiments, the cap analog encodes for adenovirus, alphavirus, calicivirus (e.g., a calicivirus capsid antigen), coronavirus polypeptides, distemper virus, Ebola virus polypeptides, enterovirus, flavivirus, hepatitis virus (AE), herpesvirus, infectious peritonitis virus, leukemia virus, Marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, and rotavirus. In certain implementations, the RNA encodes for SARS-CoV-2, HPV (e.g., E6 and/or E7 from HPV16 and/or HPV18), or influenza (e.g., influenza hemagglutinin (HA).

In some embodiments, the combined delivery of two or more particular cap analogs as disclosed herein together may be especially useful for therapeutic applications. For example, in some embodiments, the one or more cap analogs includes a combination of sgRNA (single guide RNA) as a CRISPR sequence and mRNA encoding Cas9. In still further embodiments, the cap analogs may also be complexed with proteins such as with the CRISPR/Cas9 ribonucleoprotein complex. In some cases, the cap analogs complex with one or more of a nucleic acid selected from DNA and RNA (e.g., an antigenic RNA and adjuvanting DNA, such as CpG).

A pharmaceutically effective amount of the pharmaceutical formulation and/or the cap analog (e.g., comprising RNA) may be administered to a cell (such as a mammalian cell) in vitro, ex vivo or in vivo for therapeutic or diagnosis purposes. The cap analog comprising RNA may be translated into a polypeptide or a protein. In some instances, the translation efficiency is greater than about 75%, such as about 80%, about 85%, about 90%, or about 95%.

The present disclosure also provides kits for preparing capped RNAs, comprising the cap analogs, RNA polymerases, NTPs, and DNA templates. The kits may also comprise additional enzymes, a reaction buffer, and/or instructions to conduct the method, such as incubation temperatures.

Non-limiting examples of reaction buffers include Tris, HEPES, TAPS, MOPS, tricine, or MES.

### Drug Products, Pharmaceutical Formulations, and Modes of Administration

Also provided herein are drug products (i.e., pharmaceutical compositions) that include the cap analogs of the disclosure, and an effective amount of one or more pharmaceutically acceptable excipients. An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females. As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The cap analogs of the disclosure can be administered to a subject or patient in a therapeutically effective amount. The cap analogs can be administered alone or as part of a pharmaceutically acceptable composition or formulation. In addition, the cap analogs can be administered all at once, as for example, by a bolus injection, multiple times, or delivered substantially uniformly over a period of time. It is also noted that the dose of the cap analogs can be varied over time.

The cap analogs disclosed herein and other pharmaceutically active compounds, if desired, can be administered to a subject or patient by any suitable route, e.g., orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g., eneteral or parenteral). All methods that can be used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions for parenteral administrations are administered in a sterile medium. Depending on the vehicle used and concentration the concentration of the drug in the formulation, the parenteral formulation can either be a suspension or a solution containing dissolved cap analog. Adjuvants such as local anesthetics, preservatives and buffering agents can also be added to parenteral compositions.

When the cap analogs of the disclosure are used as vaccines, they may comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the host's immune response to another antigen when administered conjointly with that antigen. Specifically, the terms “adjuvant” and “immunologic adjuvant” are used interchangeably in the present disclosure. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines. Adjuvants may be aluminum based adjuvants including but not limiting to aluminum hydroxide and aluminum phosphate; saponins such as steroid saponins and triterpenoid saponins; bacterial flagellin and some cytokines such as GM-CSF. Adjuvants selection may depend on antigens, vaccines and routes of administrations.

In some implementations, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures. In one example, the composition further comprises pidotimod as an adjuvant. In another example, the composition further comprises CpG as an adjuvant.

The cap analogs of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Use

The cap analogs disclosed herein can be delivered to a cell. Accordingly, disclosed herein are methods of delivering a cap analog, such as a nucleic acid (e.g., RNA) to a cell comprising contacting the cell with a cap analog or pharmaceutical composition disclosed herein. In some embodiments, the cell can be contacted in vitro. In some embodiments wherein the cell is contacted in vitro, the cell is a HeLa cell. In other embodiments wherein the cell is contacted in vivo, the cap analogs of the present disclosure are administered to a mammalian subject. A mammalian subject may include but is not limited to a human or a mouse subject. In yet other embodiments wherein the cell is contacted ex vivo, the cell is obtained from a human or mouse subject. In some cases, the cell is a tumor cell. In some cases, the cell is a muscle cell.

In some embodiments, the one or more cap analogs may be delivered for therapeutic uses. Non-limiting therapeutic uses include cancer, infectious diseases, autoimmune disorders, and neurological disorders. In certain implementations, the complex comprising the multicomponent delivery system and the polyanionic cargo compound is used as a vaccine. Genetic vaccination, or the administration of nucleic acid molecules (e.g., RNA) to a patient and subsequent transcription and/or translation of the encoded genetic information, is useful in the treatment and/or the prevention of inherited genetic diseases but also autoimmune diseases, infectious diseases, cancerous or tumor-related diseases as well as inflammatory diseases. Genetic vaccination is useful for treating or preventing viral infections, such as those caused by a coronavirus. In the case of coronaviruses, the vaccine target of the majority of these entities is the coronavirus' spike (S) protein, a heavily glycosylated trimeric class I fusion protein that coats the outside of the virus and is responsible for host cell entry. The S protein of SARS-CoV-2 shares high structural homology with SARS-CoV-1 and contains several subunits vital for viral entry into host cells through the angiotensin converting enzyme 2 (ACE2) receptor, including the S1 domain, the S2 domain, and the receptor binding domain (RBD). Thus, the S protein and its subunits, as well as accessible peptide sequences within these domains, are attractive vaccine antigen targets. Further, genetic vaccination is particularly use in the treatment of cancer because cancer cells express antigens, tumors are generally not readily recognized and eliminated by the host, as evidenced by the development of disease.

Vaccines. The cap analogs of the disclosure are also useful as vaccines, in which the cap analog is an RNA that may encode an immunogen, antigen or neoantigen. The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite. The cap analogs of the disclosure have been found to induce immune responses when one or more of the cap analogs encodes a viral peptide (e.g. a viral polypeptide), a viral protein, or functional fragment of the foregoing. For example, cap analogs comprising either DV-140-F2 or DV-140-F6/17 complexed with mRNA encoding the HPV E6/E7 (e.g., from HPV 16 and/or HPV 18) oncogene, a construct of the SARS-CoV spike (S) protein, and/or influenza hemagglutinin (HA) elicited strong humoral and cellular immune responses.

Thus, the disclosure includes methods for inducing an immune response in a subject in need thereof, comprising administering to the subject an effective amount of the cap analogs (e.g., formulated as an antigenic composition) of the disclosure. Also disclosed herein is a method of treating a viral infection in a subject in need thereof, comprising administering to the subject an effective amount of the cap analogs of the disclosure. In some embodiments, the administering is by intramuscular, intratumoral, intravenous, intraperitoneal, or subcutaneous delivery.

In various embodiments, administering the cap analogs of the disclosure (e.g., formulated as a composition, pharmaceutical formulation, or antigenic composition) to a subject can result in an increase in the amount of antibodies (e.g., neutralizing antibodies) against the viral antigen that is produced in the subject relative to the amount of antibodies that is produced in a subject who was not administered the cap analogs. In some embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the cap analogs comprising RNA that encodes a viral antigen as disclosed herein generates an immune response that recognizes, and preferably ameliorates and/or neutralizes, a viral infection as described herein. Methods for assessing antibody responses after administration of an antigenic composition (immunization or vaccination) are known in the art and/or described herein. In some implementations, the immune response comprises a T cell-mediated response (e.g., peptide-specific response such as a proliferative response or a cytokine response). In some implementations, the immune response comprises both a B cell and a T cell response. Antigenic compositions can be administered in a number of suitable ways, such as intramuscular injection, intratumoral injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Cancer. Various cancers (e.g., cervical cancer) may be treated with the cap analogs of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

As a non-limiting example, the cancer which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymomal thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In some embodiments, the cap analogs of the disclosure are used to treat a cancer selected from the group consisting of cervical cancer, head and neck cancer, B-cell lymphoma, T-cell lymphoma, prostate cancer, and lung cancer. In some embodiments, the cap analogs can be used to treat cervical cancer.

Infectious Diseases. In some embodiments, the cap analogs of the present disclosure are used to treat infectious diseases, such as microbial infection, e.g., a viral infection, a bacterial infection, a fungal infection, or a parasitic infection. Non-limiting examples of infectious diseases include hepatitis (such as HBV infection or HCV infection), RSV, influenza, adenovirus, rhinovirus, coronavirus, or other viral infections.

Autoimmune diseases. Various autoimmune diseases and autoimmune-related diseases may be treated with cap analogs of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed ryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Neurological diseases. Various neurological diseases may be treated with the cap analogs of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Shortlasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### EXAMPLES

The presently disclosed subject matter will be better understood by reference to the following Examples, which are provided as exemplary, and not by way of limitation. In Table 2, Cap 1, Cap 2, and Cap 3 are comparator compounds to the compounds of the disclosure.

TABLE 1

| Compound ID | Structure |
|---|---|
| Compound 1 (Compd 1) | 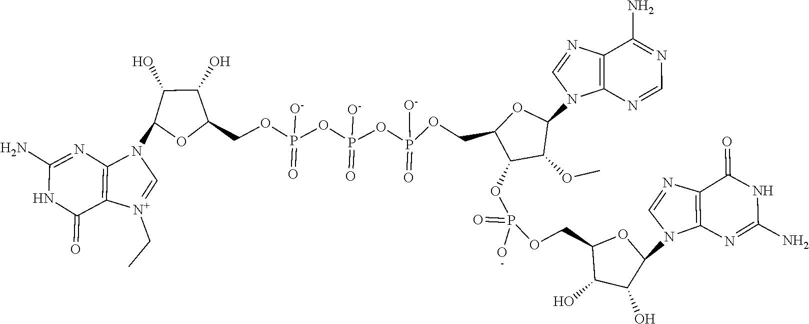 [SMILES: CC[n+]1cn([C@@H]2O[C@H](COP(=O)([O-])OP(=O)([O-])OP(=O)([O-])OC[C@H]3O[C@@H](n4cnc5c(N)ncnc54)[C@H](OC)[C@@H]3OP(=O)(O)OC[C@H]3O[C@@H](n4cnc5c(=O)[nH]c(N)nc54)[C@H](O)[C@@H]3O)[C@@H](O)[C@H]2O)c2nc(N)[nH]c(=O)c21] |
| Compound 2 (Compd 2) | 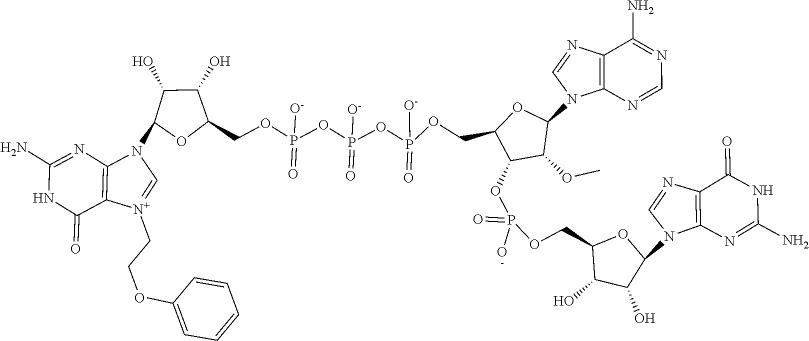 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)([O-])OP(=O)([O-])OP(=O)([O-])OC[C@H]2O[C@@H](n3c[n+](CCOc4ccccc4)c4c(=O)[nH]c(N)nc43)[C@H](O)[C@@H]2O)O[C@H]1n1cnc2c(N)ncnc21] |
| Compound 3 (Compd 3) | 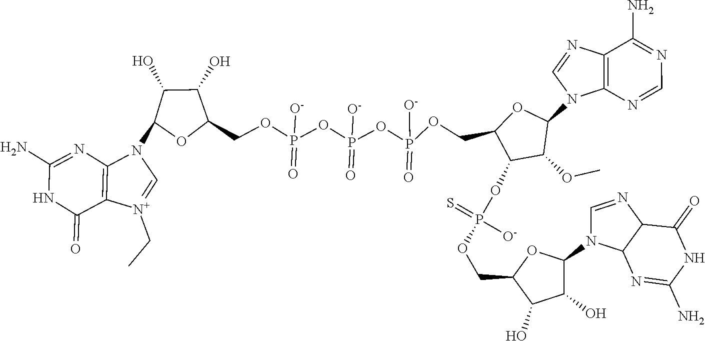 [SMILES: CC[n+]1cn([C@@H]2O[C@H](COP(=O)([O-])OP(=O)([O-])OP(=O)([O-])OC[C@H]3O[C@@H](n4cnc5c(N)ncnc54)[C@H](OC)[C@@H]3O[P@@]([O-])(=S)OC[C@H]3O[C@@H](N4C=NC5C(=O)NC(N)=NC54)[C@H](O)[C@@H]3O)[C@@H](O)[C@H]2O)c2nc(N)[nH]c(=O)c21] |
| Compound 4 (Compd 4) | 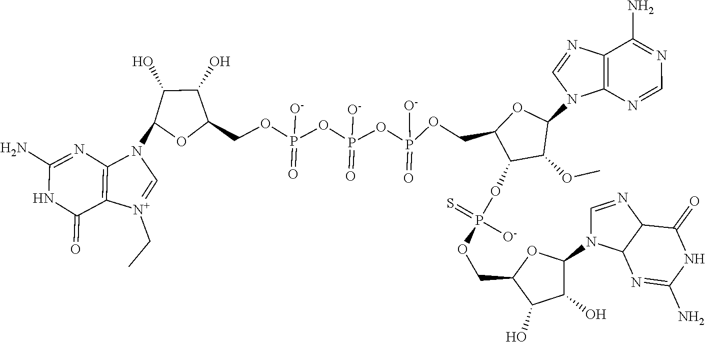 [SMILES: CC[n+]1cn([C@@H]2O[C@H](COP(=O)([O-])OP(=O)([O-])OP(=O)([O-])OC[C@H]3O[C@@H](n4cnc5c(N)ncnc54)[C@H](OC)[C@@H]3O[P@]([O-])(=S)OC[C@H]3O[C@@H](N4C=NC5C(=O)NC(N)=NC54)[C@H](O)[C@@H]3O)[C@@H](O)[C@H]2O)c2nc(N)[nH]c(=O)c21] |

TABLE 2

| Compound ID | Structure |
|---|---|
| Cap 1 | 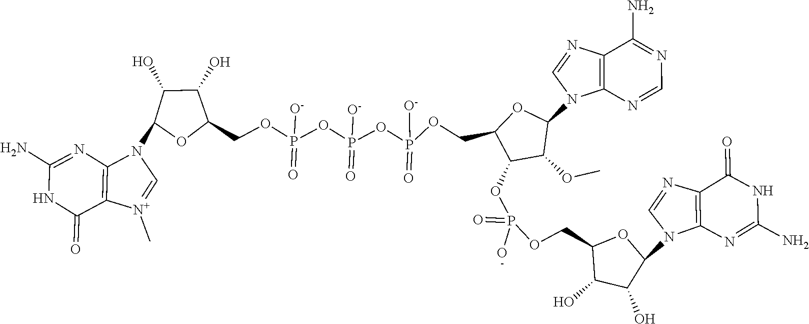 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)([O-])OP(=O)([O-])OP(=O)([O-])OC[C@H]2O[C@@H](n3c[n+](C)c4c(=O)[nH]c(N)nc43)[C@H](O)[C@@H]2O)O[C@H]1n1cnc2c(N)ncnc21] |
| Cap 2 | 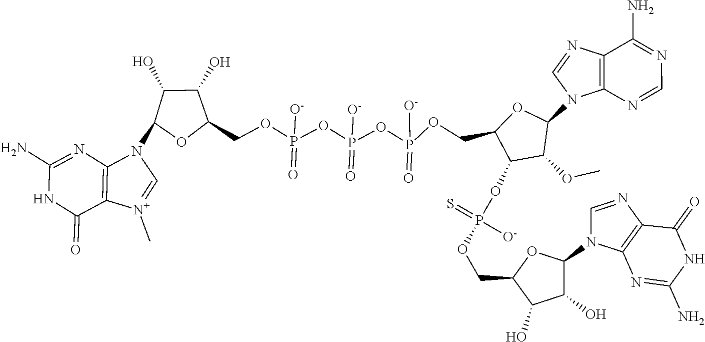 [SMILES: CO[C@@H]1[C@H](O[P@@]([O-])(=S)OC[C@H]2O[C@@H](n3cnc4c(=O)[nH]c(N)nc43)[C@H](O)[C@@H]2O)[C@@H](COP(=O)([O-])OP(=O)([O-])OP(=O)([O-])OC[C@H]2O[C@@H](n3c[n+](C)c4c(=O)[nH]c(N)nc43)[C@H](O)[C@@H]2O)O[C@H]1n1cnc2c(N)ncnc21] |
| Cap 3 | 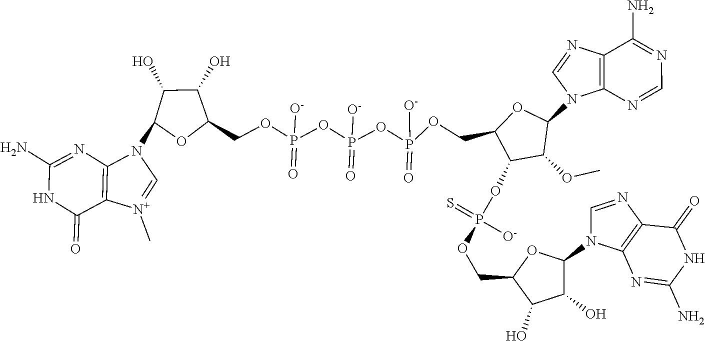 [SMILES: CO[C@@H]1[C@H](O[P@]([O-])(=S)OC[C@H]2O[C@@H](n3cnc4c(=O)[nH]c(N)nc43)[C@H](O)[C@@H]2O)[C@@H](COP(=O)([O-])OP(=O)([O-])OP(=O)([O-])OC[C@H]2O[C@@H](n3c[n+](C)c4c(=O)[nH]c(N)nc43)[C@H](O)[C@@H]2O)O[C@H]1n1cnc2c(N)ncnc21] |

### Materials and Methods

In Vitro Transcription of mRNAs Containing Cap Analogs

mRNA was transcribed from DNA template encoding Lucia using HiScribe T7 RNA synthesis kit (New England Biolabs, Ipswich, MA, USA). N¹-Methylpseudo-UTP (TniLink, San Diego, CA, USA) was used in replaced of UTP for the reaction. For 5′ capping of the mRNA, the cap analog compounds (e.g., Compounds 1-4) described herein, or CleanCap (TriLink, San Diego, CA, USA), were added to the IVT reaction, as indicated. The reactions were performed for 4 hours at 37° C.

Purification of the transcript was performed by TURBO DNase digestion (ThermoFisher Scientific, Waltham, MA, USA) for 30 min at 37° C. followed by RNAClean XP beads (Beckman Coulter) clean up. The RNA bound beads were washed 3 times with freshly prepared 70% ethanol, air dried for 15 minutes followed by elution in 200 μL of nuclease free water. The purified mRNA was quantified using nanodrop (ThermoFisher Scientific, Waltham, MA, USA) and analyzed using Tapestation (Agilent, Santa Clara, CA, USA).

In Vitro Expression of mRNAs Containing Cap Analogs

Lucia coding mRNA containing the cap analog compounds (e.g., Compounds 1-4) described herein, or CleanCap (TriLink, San Diego, CA, USA), were transfected into HEK 293 cells using Lipofectamine MessengerMAX (ThermoFisher Scientific, Waltham, MA, USA), according to manufacturer's protocol.

Lucia Luciferase activity was measured at 2, 4, 6, 24, 48 and 72 hours post transfection. At each time point, 5 μL of media was removed from each well to mix with QUANTI-Luc (InvivoGen, San Diego, CA, USA) and measured immediately using a plate reader (Molecular Devices, San Jose, CA, USA). Fresh media containing DMEM with 10% FBS and 50 U/mL Penicillin/Streptomycin were replaced in each well after each time point. The experiment was performed in triplicate in 96 well plate with a seeding density of 1e4 cells per well, and the cells were kept at 37° C. incubator supplied with 5% CO₂.

In Vivo Expression of mRNAs Containing Cap Analogs

Candidate RNAs were formulated using proprietary lipid-based delivery vehicles (e.g., as described in WO 2020/069442 and WO 2023/014931) and administered to Balb/c mice at a dose of 15 μg per mouse. In life serum draws were conducted via submandibular vein at selected time points starting 2 hours post-dose. Serum was diluted 5 μL into 50 mL of QUANTI-Luc reagent (InvivoGen, San Diego, CA, USA) and measured immediately using a plate reader (Molecular Devices, San Jose, CA, USA).

### Example 1: Synthesis of Compound 1 (Compd 1)

### Step-1: Synthesis of N-(9-((2R,3R,4R,5R)-4-hydroxy-5-(hydroxymethyl)-3-methoxytetrahydrofuran-2-yl)-9H-purin-6-yl)benzamide (1)

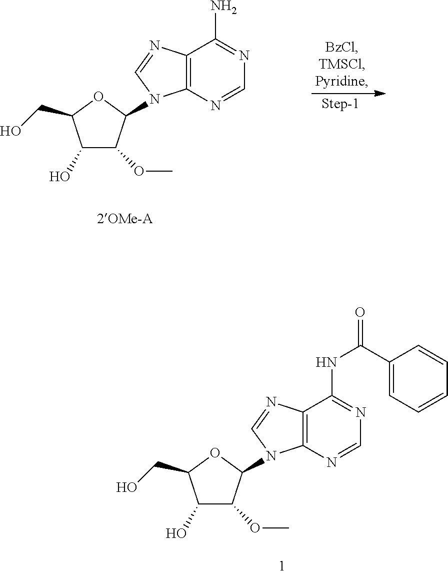 [SMILES: CO[C@@H]1[C@H](O)[C@@H](CO)O[C@H]1n1cnc2c(N)ncnc21 ; CO[C@@H]1[C@H](O)[C@@H](CO)O[C@H]1n1cnc2c(NC(=O)c3ccccc3)ncnc21]

To a stirred solution of 2′-OMe-A (50 g, 0.178 mol, 1 eq) in dry distilled pyridine (500 mL) at 0° C. was added TMS-CI (224 mL, 1.78 mol, 10 eq) by drop wise then stirred at room temperature. After 2 h, distilled benzoyl chloride (30 mL, 0.26 mmol, 1.5 eq) was added to the reaction mixture at 0° C. then allowed to warm to room temperature (25° C.) over 2 h. Reaction progress was monitored by TLC and LCMS. Reaction mixture was cooled to 0° C., quenched with ice cold water (200 mL), stirred for 30 min, then aq. NH₄OH (100 mL) was added and the mixture was stirred for 30 min and distilled to afford semi-pure material. This was further purified by SiO₂ (60-120 mesh) column chromatography by elution with 0-5% Methanol (MeOH) in Dichloromethane (DCM) to obtain pure Intermediate 1. TLC system: MeOH/DCM (10:90), R_{f} value: 0.4; ¹HNMR (400 MHz, DMSO-d₆) δ 8.77-8.73 (m, 2H), 8.05 (d, J=7.6 Hz, 2H), 7.67-7.64 (m, 1H), 7.57-7.52 (m, 2H), 6.17 (d, J=5.6 Hz, 1H), 4.45-4.39 (m, 2H), 4.02-4.01 (m, 1H), 3.72-3.69 (m, 1H), 3.62-3.59 (m, 1H), 3.37 (s, 3H); LCMS indicates 73% with 386.3 (M+H)⁺.

### Step-2: Synthesis of N-(9-((2R,3R,4R,5R)-5-((bis(4-methoxyphenyl)(phenyl)methoxy)methyl)-4-hydroxy-3-methoxytetrahydrofuran-2-yl)-9H-purin-6-yl)benzamide (Int-2)

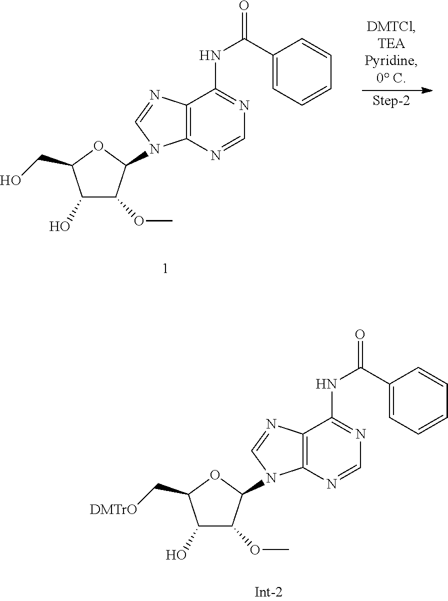

To the stirred solution of Intermediate 1 (42 g, 109.1 mmol, 1 eq) (co-distilled with dry toluene for 3 times) in dry distilled Pyridine (85 mL) at 0° C. was added dry distilled TEA (22.7 mL, 163.6 mmol, 1.5 eq) then added lot wise DMT-CI (74 g, 218.2 mmol, 2 eq) and allowed to room temperature for 5 h. After completion of starting material by TLC, reaction mixture was diluted with cold DM water (1 L) and extracted into EtOAC (2×350 mL). The combined organic layers were washed with brine solution (300 mL) dried over anhydrous sodium sulfate, filtered and concentrated under reduced pressure to afford semi-pure product. This was further purified by SiO₂ (60-120 mesh) column chromatography by elution with 70-80% of EtOAc in Hexanes to afford Int-2. TLC system:EtOAc/Hexane (70:30), R_{f} value: 0.5; ¹HNMR (400 MHz, DMSO-d₆) δ 11.23 (s, 1H), 8.69 (s, 1H), 8.61 (s, 1H), 8.06-8.04 (m, 2H), 7.67-7.63 (m, 1H), 7.57-7.54 (m, 2H), 7.37-7.35 (m, 2H), 7.28-7.18 (m, 7H), 6.86-6.83 (m, 4H), 6.19 (d, J=4.8 Hz, 1H), 5.35 (d, J=6.0 Hz, 1H), 4.56-4.53 (m, 1H), 4.50-4.46 (m, 1H), 4.13-4.10 (m, 1H), 3.72 (s, 6H), 3.40 (s, 3H), 3.27-3.25 (m, 2H); LCMS 83% with m/z: 688.3 (M+H)⁺.

### Step-3: Synthesis of N-(9-((2R,3R,4R,5R)-5-((bis(4-methoxyphenyl)(phenyl)methoxy)methyl)-3-methoxy-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-9H-purin-6-yl)benzamide (Int-6)

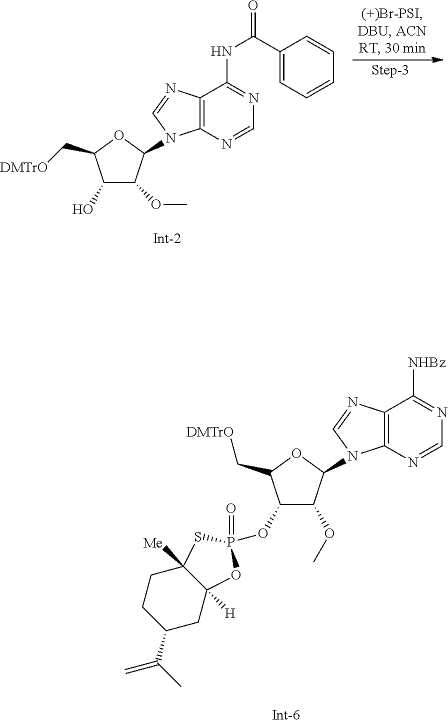

To the stirred solution of Int-2 (10 g, 14.5 mmol, 1 eq) and (+)Br-PSI reagent (8.2 g, 18.85 mmol, 1.3 eq) in dry distilled ACN (100 mL) at 0° C. was added a solution of DBU (4.47 g, 29.0 mmol, 1.2 eq) in acetonitrile (5 mL) and continued the stirring for 45 min. After complete consumption of PSI monitored by TLC, the reaction mixture was distilled to reduce acetonitrile volume up to 20 mL. The mixture was purified by silica gel (100-200 mesh) column chromatography eluting with 30% EtOAc in Hexane to afford Int-6. TLC system:EtOAc/Hexane (30:70), R_{f} value: ^{˜}0.7; ¹HNMR (400 MHz, DMSO-d₆) δ 11.25 (s, 1H), 8.68 (s, 1H), 8.60 (s, 1H), 8.05 (d, J=7.2 Hz, 2H), 7.67-7.63 (m, 1H), 7.58-7.54 (m, 2H), 7.40-7.39 (m, 2H), 7.28-7.19 (m, 7H), 6.86-6.83 (m, 4H), 6.13-6.12 (m, 1H), 5.53-5.49 (m, 1H), 5.05-5.02 (m, 1H), 4.95-4.85 (m, 2H), 4.41-4.34 (m, 3H), 3.73 (s, 6H), 3.39-3.37 (m, 1H), 3.34 (s, 3H), 2.58-2.51 (m, 2H), 2.23-2.15 (m, 1H), 1.93-1.87 (m, 2H), 1.83-1.77 (m, 2H), 1.73 (s, 3H), 1.66 (s, 3H); LCMS 77% with m/z: 933.9 (M+H)⁺

### Step-10: Synthesis of N-(9-((2R,3R,4S,5R)-5-((bis(4-methoxyphenyl)(phenyl)methoxy)methyl)-3,4-dihydroxytetrahydrofuran-2-yl)-6-oxo-6,9-dihydro-1H-purin-2-yl)benzamide (4A)

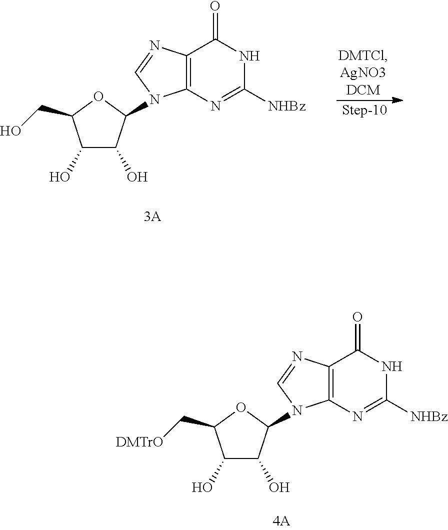

To a stirred solution of Compound-3A (10 g, 25.83 mmol, 1 eq) in DCM (50 mL, 5 vol) at 0° C. was added AgNO₃ (8.7 g, 51.67 mmol, 2 eq) and lot wise addition of DMT-CI (17.4 g, 51.67 mmol, 2 eq) then stirred at room temperature for 5 h. After complete consumption of starting material by TLC, reaction mixture was diluted with cold water (500 mL) and extracted with DCM (2×300 mL). The combined organic layers were washed with brine solution (250 mL) dried over anhydrous sodium sulfate and concentrated under reduced pressure to afford semi-pure compound. Normal phase SiO₂ column chromatography purification by eluting with 3-5% MeOH in DCM afforded compound-4A. TLC system:MeOH/DCM (10:90), R_{f} value: 0.5; LCMS m/z: 690.0 (M+H)⁺.

### Synthesis of (2R,3R,4R,5R)-2-(2-benzamido-6-oxo-1,6-dihydro-9H-purin-9-yl)-5-((bis(4-methoxyphenyl)(phenyl)methoxy)methyl)tetrahydrofuran-3,4-diyl bis(2-methylpropanoate) (5A)

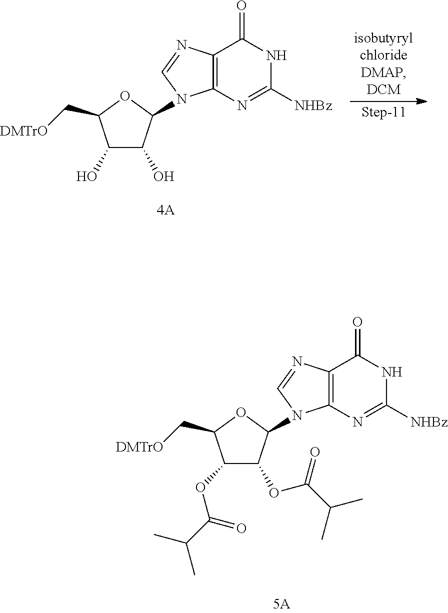

To the stirred solution of compound-4A (15 g, 21.77 mmol, 1 eq) and DMAP (3.9 g, 32.65 mmol, 1.5 eq) in DCM (450 mL, 30 vol) was added isobutyryl chloride (3.4 mL, 32.65 mmol, 1.5 eq) by drop wise addition at 0° C. then allowed to stir at room temperature for 1 h. After completion of reaction by TLC, reaction mixture was cooled to 0° C. quenched with ice cold water (500 mL) and extracted with DCM (2×300 mL) washed with brine solution (250 mL). The organic layer was dried over anhydrous sodium sulfate and concentrated under reduced pressure to afford semi-pure compound. Normal phase SiO₂ column purification by eluting with 70% EtOAc in Hexanes afforded compound-5A. TLC system:MeOH/DCM (05:95), R_{f} value: 0.4; LCMS m/z: 828.0 (M+H)⁺.

### Step-12: Synthesis of (2R,3R,4R,5R)-2-(2-benzamido-6-oxo-1,6-dihydro-9H-purin-9-yl)-5-(hydroxymethyl)tetrahydrofuran-3,4-diyl bis(2-methylpropanoate) (Nuc-C2)

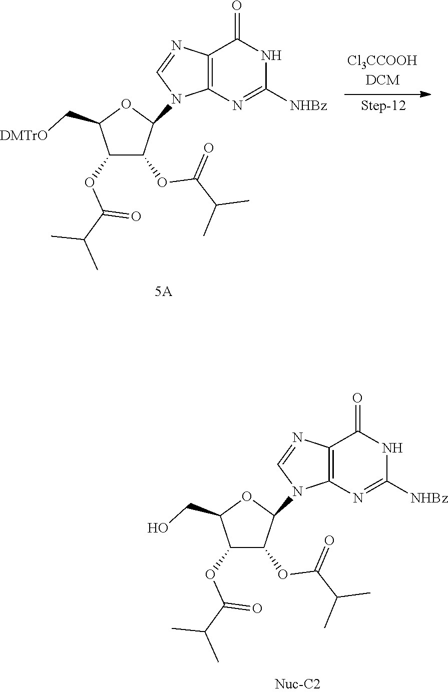

To the compound-5A (20 g, 71.17 mmol, 1 eq) was added a solution of Cl₃CCOOH in DCM (5 g/50 mL) by drop wise addition at 0° C., which was then allowed to stir at room temperature for 2 h. After completion of reaction by TLC, reaction mixture was quenched with aq. NaHCO₃ and extracted with DCM (2×200 mL) washed with brine solution (350 mL). The organic layer was dried over anhydrous sodium sulfate and concentrated under reduced pressure to afford semi-pure compound. Normal phase SiO₂ column purification by eluting with 5% MeOH in DCM afforded Nuc-C2. TLC system:MeOH/DCM (05:95), R_{f} value: 0.4; ¹HNMR (400 MHz, DMSO-d₆) δ 12.35 (s, 1H), 11.89 (s, 1H), 8.34 (s, 1H), 8.04-8.01 (m, 2H), 7.71-7.66 (m, 1H), 7.59-7.55 (m, 2H), 6.13 (d, J=7.2 Hz, 1H), 5.84-5.81 (m, 1H), 5.52-5.50 (m, 1H), 5.39 (t, 1H), 4.23-4.21 (m, 1H), 4.10-4.07 (m, 1H), 3.72-3.68 (m, 2H), 2.65-2.61 (m, 1H), 1.16 (dd, J=6 Hz, 2.8 Hz, 6H), 1.01 (dd, J=6.8 Hz, 16 Hz, 6H); LCMS m/z: 526.0 (M+H)⁺

### Step-4: Synthesis of (2R,3R,4R,5R)-2-(2-benzamido-6-oxo-1,6-dihydro-9H-purin-9-yl)-5-((((S)-(((2R,3R,4R,5R)-5-(6-benzamido-9H-purin-9-yl)-2-((bis(4-methoxyphenyl)(phenyl)methoxy)methyl)-4-methoxytetrahydrofuran-3-yl)oxy)(hydroxy)phosphorothioyl)oxy)methyl)tetrahydrofuran-3,4-diyl bis(2-methylpropanoate) (22)

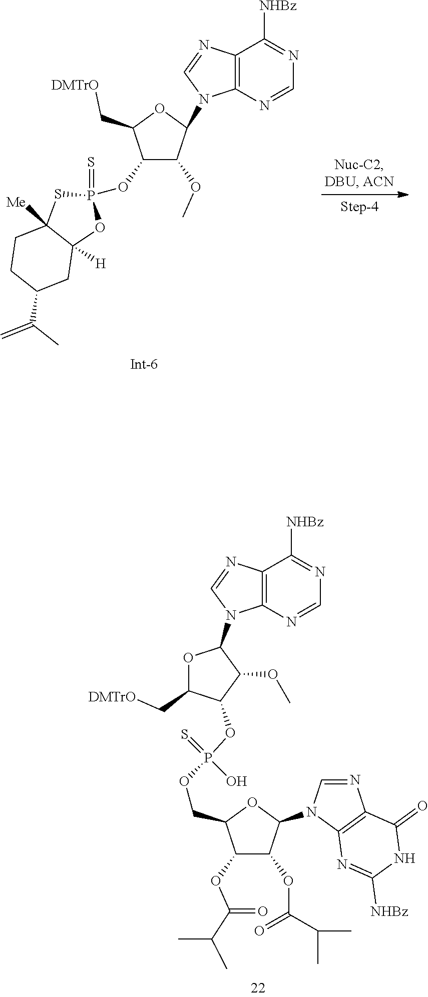

To the stirred solution of Int-6 (9 g, 9.64 mmol, 1 eq) (co-distilled with dry ACN) and Nuc-C2 (7.6 g, 14.46 mmol, 1.5 eq) (co-distilled with dry ACN) in dry distilled ACN (70 mL) was added a solution of dry distilled DBU (1.45 g, 9.64 mmol, 1 eq) in acetonitrile (15 mL) at 0° C. by drop wise and continued the stirring for 40 min. After complete consumption of starting material the reaction mixture was distilled off then triturated with diethyl ether to afford compound-22. LCMS indicates 34% with m/z: 1292.9 (M+H)⁺.

### Step-5: Synthesis of (2R,3R,4R,5R)-2-(2-benzamido-6-oxo-1,6-dihydro-9H-purin-9-yl)-5-((((((2R,3R,4R,5R)-5-(6-benzamido-9H-purin-9-yl)-2-(hydroxymethyl)-4-methoxytetrahydrofuran-3-yl)oxy)(hydroxy)phosphoryl)oxy)methyl)tetrahydrofuran-3,4-diyl bis(2-methylpropanoate) (23)

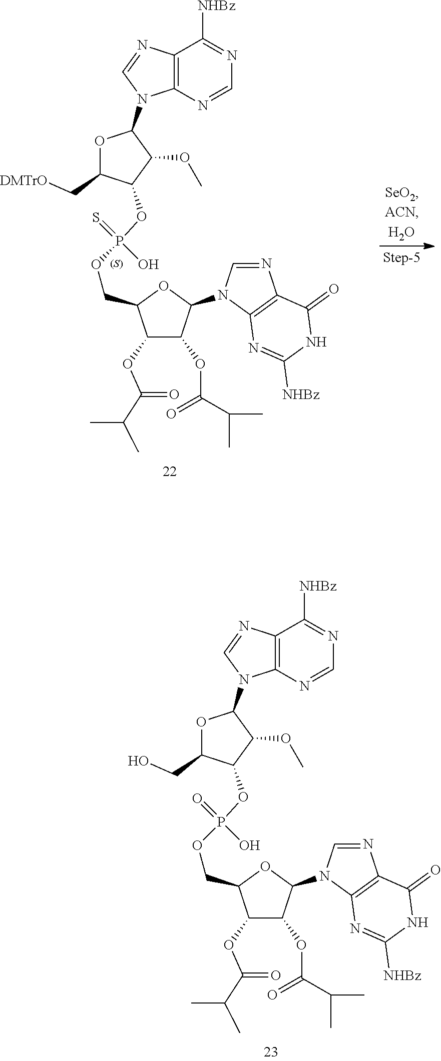

To the stirred solution of compound-22 (12 g (Semi-pure), 9.28 mmol, 1 eq) in mixture of ACN:H₂O (30 mL, 4:1 ratio) was added selenium dioxide (5.15 g, 46.43 mmol, 5 eq) at 0° C. at 25° C. and the mixture was stirred for 16 h. Reaction progress was monitored by LCMS. After completion of reaction the reaction mixture was filtered and collected, and mother liquor was distilled. This crude material was purified by C18 reverse phase column chromatography eluting with 1M ammonium bicarbonate buffer solution and 25-30% of ACN. Collected pure fraction was lyophilized to afford compound-23. LCMS 80% with m/z: 974.8 (M+H)⁺.

### Step-6: Synthesis of (2R,3R,4R,5R)-2-(2-benzamido-6-oxo-1,6-dihydro-9H-purin-9-yl)-5-((((((2R,3R,4R,5R)-5-(6-benzamido-9H-purin-9-yl)-4-methoxy-2-((phosphonooxy)methyl)tetrahydrofuran-3-yl)oxy)(hydroxy)phosphoryl)oxy)methyl)tetrahydrofuran-3,4-diyl bis(2-methylpropanoate) (24)

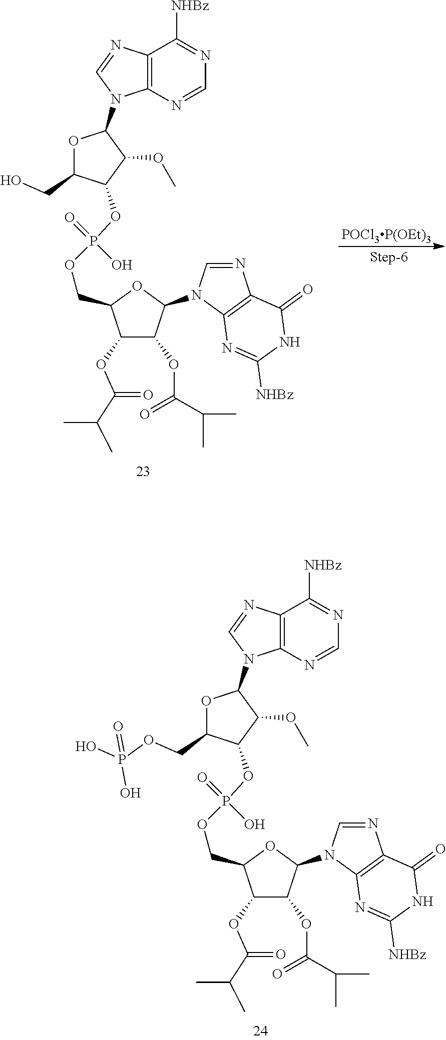

To the stirred solution of compound-23 (2.5 g, 2.56 mmol, 1 eq) in distilled Trimethyl phosphate (2.99 mL, 25.66 mmol, 10 eq) the clear solution was observed after addition of distilled phosphoryl chloride (1.9 mL, 20.48 mmol, 8 eq) at 0° C. for 4 h. Reaction progress was monitored by LCMS, after complete consumption of starting material, the reaction mixture was quenched with 1M TEAB then lyophilized. This material was purified by C18 reverse phase column chromatography eluting with 1M aq. ammonium bicarbonate and 20-25% ACN. The collected pure fraction was lyophilized to afford compound 24 as triethyl ammonium salt. LCMS 84% with m/z: 1054.9 (M+H)⁺.

### Synthesis of 2-amino-9-((2R,3R,4S,5R)-3,4-dihydroxy-5-(((hydroxy(phosphonooxy)phosphoryl) oxy)methyl)tetrahydrofuran-2-yl)-7-ethyl-6-oxo-6,9-dihydro-1H-purin-7-ium (Et7GDP)

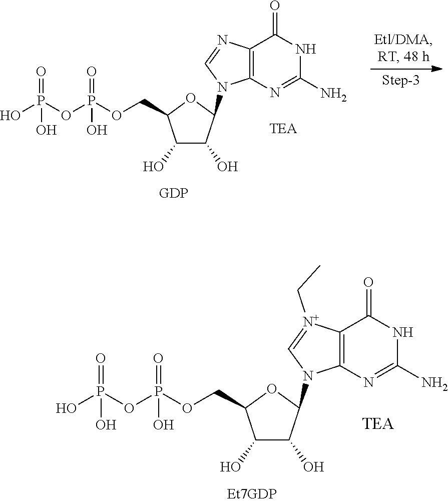 [SMILES: Nc1nc2c(ncn2[C@@H]2O[C@H](COP(=O)(O)OP(=O)(O)O)[C@@H](O)[C@H]2O)c(=O)[nH]1 ; CC[n+]1cn([C@@H]2O[C@H](COP(=O)(O)OP(=O)(O)O)[C@@H](O)[C@H]2O)c2nc(N)[nH]c(=O)c21]

To a stirred solution of triethyl amine salt of guanosine-5′-diphosphate (7 g, 15.08 mmol, 1 eq) in dry dimethyl acetamide (35 mL, 5 vol) was added ethyl iodide (6.5 mL, 79.8 mmol, 5 eq) then stirred at room temperature for 7 days. After completion of reaction monitored by LCMS/1HNMR, to reaction mixture was added diethyl ether and the formed solid was filtered then washed with acetone and dried. The obtained crude solid was purified by DEAE Sephadex chromatography by eluting with 12-15% 1M aq. TEAB and water. The collected desired fraction was lyophilized to obtain semi-pure compound which was further purified twice by prep-HPLC using C18 column to afford Et7GDP. ¹HNMR (400 MHz, D₂O) δ 9.23 (S, 1H), 6.04 (d, J=3.2 Hz, 1H), 4.61-4.52 (m, 3H), 4.45-4.35 (m, 2H), 4.27-4.13 (m, 2H), 1.58 (t, 3H).

### Step-4: Synthesis of 2-amino-9-((2R,3R,4S,5R)-3,4-dihydroxy-5-(((hydroxy((hydroxy(1H-imidazol-1-yl)phosphoryl)oxy)phosphoryl)oxy)methyl)tetrahydrofuran-2-yl)-7-ethyl-6-oxo-6,9-dihydro-1H-purin-7-ium (Et7GDP-Im)

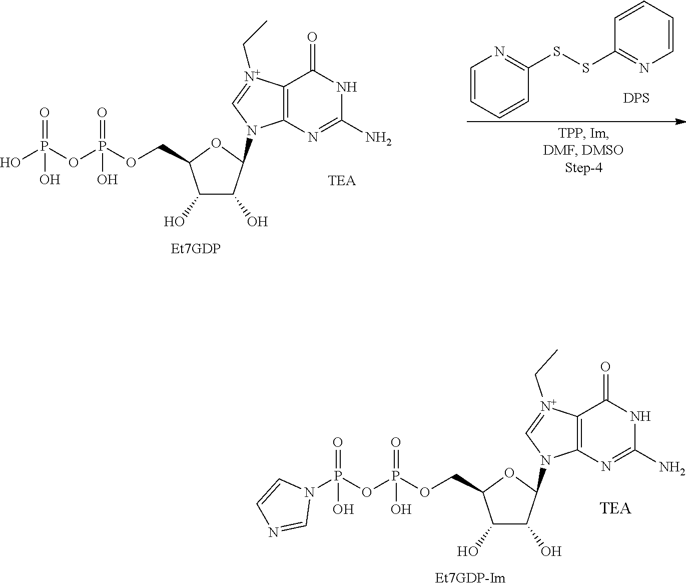

To the solution of Et7GDP (650 mg, 1.35 mmol, 1 eq), TPP (4.17 g, 6.79 mmol, 5 eq), Imidazole (920 mg, 13.5 mmol, 10 eq) in the mixture of DMF:DMSO (2 mL:0.3 mL) was added DPS (1.48 g, 6.79 mmol, 5 eq) at 0° C. then allowed to stir at room temperature for 2 h. After complete consumption of starting material as monitored by LCMS, the reaction mixture was cooled to 0° C. then added chilled sodium perchlorate in acetone solution. The formed precipitate was collected by centrifuge and washed thoroughly with acetone then dried. 450 mg of Et7GDP-Im was isolated as an off-white solid (yield: 76%); LCMS m/z: 522.0 (M+H)⁺.

### Step-1:2-amino-9-((2R,3R,4S,5R)-5-((((((((((2R,3R,4R,5R)-3-(((((2R,3R,4R,5R)-5-(2-benzamido-6-oxo-1,6-dihydro-9H-purin-9-yl)-3,4-bis(isobutyryloxy)tetrahydrofuran-2-yl)methoxy)(hydroxy)phosphoryl)oxy)-5-(6-benzamido-9H-purin-9-yl)-4-methoxytetrahydrofuran-2-yl)methoxy)(hydroxy)phosphoryl)oxy) (hydroxy)phosphoryl)oxy)(hydroxy)phosphoryl)oxy)methyl)-3,4-dihydroxytetrahydrofuran-2-yl)-7-ethyl-6-oxo-6,9-dihydro-1H-purin-7-ium (39)

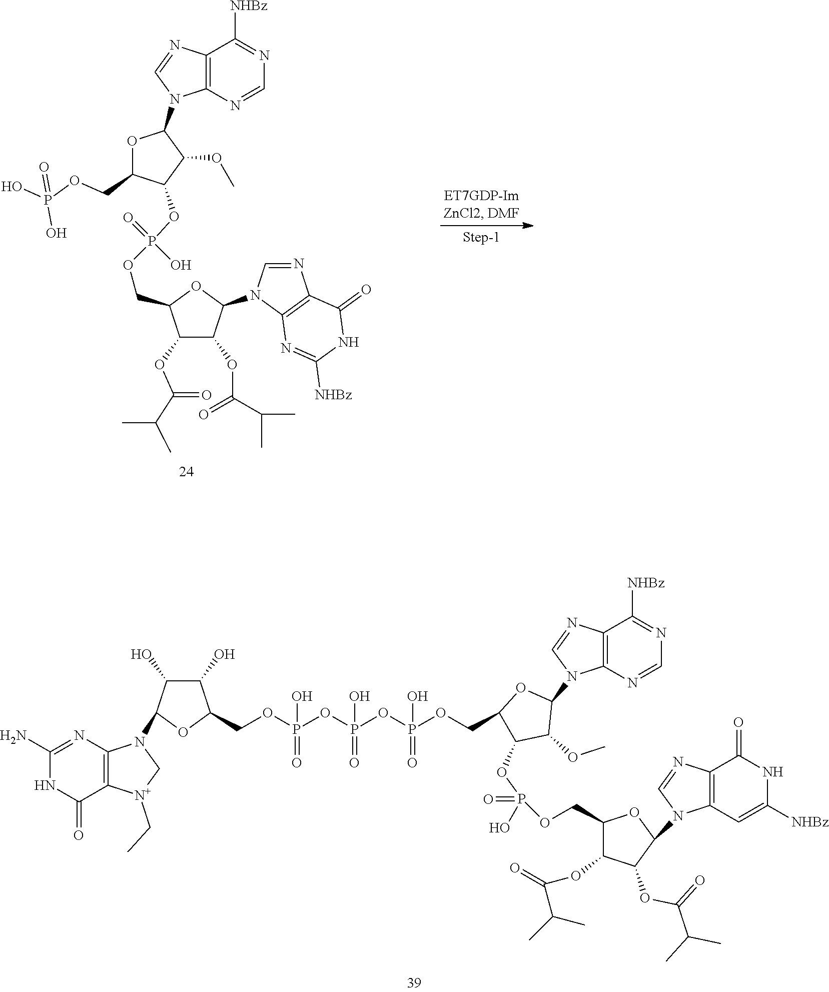

To the stirred solution of compound-24 (350 mg, 0.332 mmol, 1 eq) (co-distilled with dry toluene) and Et7GDP-Im (435 mg, 0.830 mmol, 2.5 eq) (co-distilled with dry toluene) in dry distilled DMF (10 vol) was added anhydrous ZnCl₂ (1.35 g, 9.96 mmol, 30 eq) (dried under 0.5 mmHg at 60° C. for 3 h) at 0° C. and allowed to warm to room temperature (25° C.) then stirred for 16 h.

Stage-1 (Work-up): The reaction mixture was diluted with cold water (10 mL) and EDTA solid then the obtained clear solution was lyophilized for removal of DMF. LCMS indicates compound-39 with 55%, compound-24 with 17%.

Stage-2 (DEAE Sephadex Purification): The obtained crude compound was purified by eluting through DEAE Sephadex with 30-40% ACN and 1M aq. TEAB. The collected pure fraction was lyophilized to obtain semi-pure Compound-39. LCMS indicates Compound-39 with 47% of desired m/z.

Stage-3 (C18 Purification): Above obtained semi-pure compound was further purified by C18 reverse phase Prep-HPLC eluting with 48% of ACN and 1M aq. ABC to afford pure compound-39. LCMS indicates ˜80% of desired m/z.

### Step-2: Synthesis of 2-amino-9-((2r,3r,4s,5r)-5-((((((((((2r,3r,4r,5r)-3-(((((2r,3s,4r,5r)-5-(2-amino-6- oxo-1,6-dihydro-9h-purin-9-yl)-3,4-dihydroxytetrahydrofuran-2-yl)methoxy)(hydroxy)phosphoryl)oxy)-5-(6-amino-9h-purin-9-yl)-4-methoxytetrahydrofuran-2-yl)methoxy)(hydroxy)phosphoryl)oxy) (hydroxy)phosphoryl)oxy)(hydroxy)phosphoryl)oxy)methyl)-3,4-dihydroxytetrahydrofuran-2-yl)-7-ethyl-6-oxo-6,9-dihydro-1h-purin-7-ium (Compd 1)

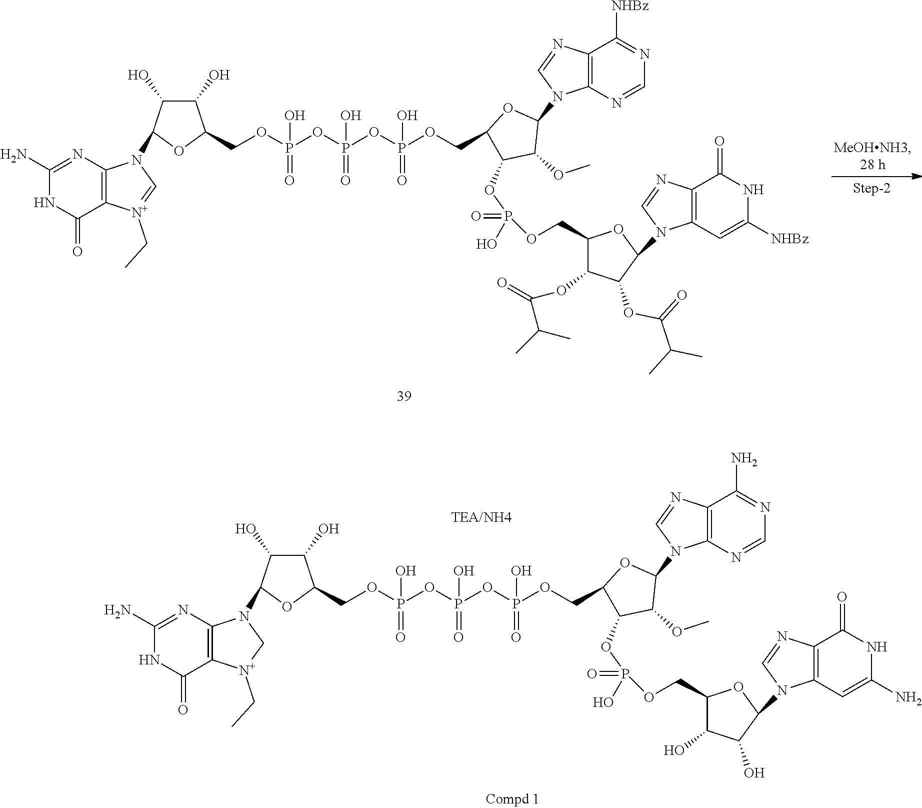

7m MEOH·NH₃ (5 vol*2 times) was added to compound-39 (65 mg, 0.043 mmol, 1 eq) in 25 ml RBF via syringe at 20° C. and stirred for 48 h at same temperature. Reaction progress was monitored by LCMS and after complete consumption of compound-65 proceeded for work-up.

Work-up: The reaction mixture was flushed with nitrogen then concentrated under reduced pressure to afford semi-pure compound Compd 1.

DEAE Sephadex Purification: The obtained semi-pure Compd 1 was further purified by eluting through DEAE Sephadex with 2-3% of ACN and 1M aq. TEAB. The collected desired fraction was lyophilized to afford semi-pure Compd 1 as TEA salt.

Prep-HPLC Purification: The obtained semi-pure Compd 1 as the TEA salt was further purified by prep-HPLC using 1M aq. ABC and mixture of 10% ACN: 30% MeOH: 60% water to afford pure Compd 1.

### Example 2: Synthesis of Compound 2 (Compd 2)

### Step-1: Synthesis of 2-phenoxyethyl methanesulfonate (A)

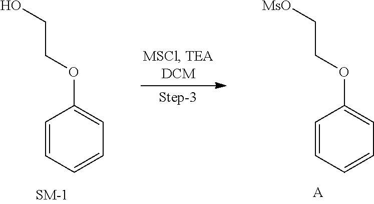

To a stirred solution of SM-1 (30 g, 0.22 mol, 1 eq) in dichloromethane (300 mL, 10 vol) were added triethylamine (41 mL, 0.32 mol, 1.5 eq) and mesyl chloride (25.1 mL, 0.32 mol, 1.5 eq) then stirred at 0° C. for 5 h. After complete consumption of starting material by TLC the reaction mixture was diluted with water and extracted with DCM then dried over sodium sulfate and concentrated under reduced pressure to afford compound-A; ¹HNMR (400 MHz, CDCl₃) δ 7.33-7.28 (m, 2H), 7.02-6.98 (m, 1H), 6.91-6.88 (m, 2H), 4.59-4.57 (m, 2H), 4.26-4.24 (m, 2H), 3.09 (s, 3H).

### Step-4: Synthesis of (2-iodoethoxy)benzene (B)

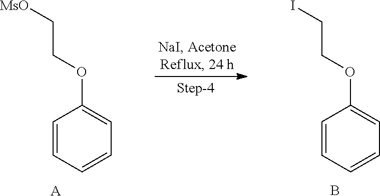

To a stirred solution of compound-A (30 g, 0.14 mol, 1 eq) in acetone (300 mL, 10 vol) was added NaI (31.2 g, 0.21 mol, 1.5 eq) then stirred at reflux temperature for 16 h. After complete consumption of starting material by TLC the reaction mixture was diluted with water and extracted with diethyl ether then dried over sodium sulfate and concentrated under reduced pressure. The obtained crude was purified by silica gel column purification to afford compound-B; ¹HNMR (400 MHz, CDCl₃) δ 7.31-7.26 (m, 2H), 7.0-6.95 (m, 1H), 6.91-6.88 (m, 2H), 4.25 (t, 2H), 3.42 (t, 2H).

### Step-5: Synthesis of 2-amino-9-((2R,3R,4S,5R)-3,4-dihydroxy-5-(((hydroxy(phosphonooxy)phosphoryl)oxy)methyl)tetrahydrofuran-2-yl)-6-oxo-7-(2-phenoxyethyl)-6,9-dihydro-1H-purin-7-ium ((CH2CH2-OPh)GDP)

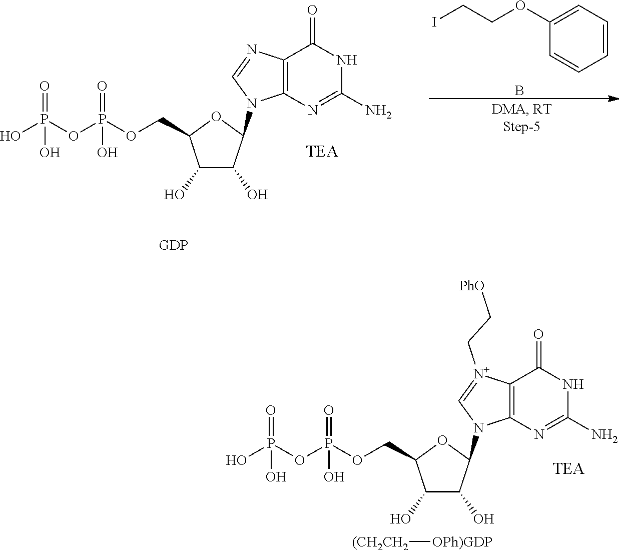

To a stirred solution of triethyl amine salt of guanosine-5′-diphosphate (3 g, 6.77 mmol, 1 eq) in dry dimethyl acetamide (6 mL, 2 vol) was added compound-B (3.34 g, 13.54 mmol, 2 eq) then stirred at 70° C. for 16 h. The reaction mixture was diluted with diethyl ether then the formed solid was washed with acetone and dried. The obtained crude solid was purified by DEAE Sephadex chromatography by eluting with 25% 1M aq. TEAB and water. The collected desired fraction was lyophilized to obtain semi-pure compound which was further purified twice by prep-HPLC using C18 column to afford (CH₂CH₂—OPh)GDP; LCMS m/z: 564.0 (M)⁺.

### Step-6: Synthesis of 2-amino-9-((2R,3R,4S,5R)-3,4-dihydroxy-5-(((hydroxy((hydroxy(1H-imidazol-1-yl)phosphoryl)oxy)phosphoryl)oxy)methyl)tetrahydrofuran-2-yl)-6-oxo-7-(2-phenoxyethyl)-6,9-dihydro-1H-purin-7-ium (CH2CH2-OPh)GDP-Im)

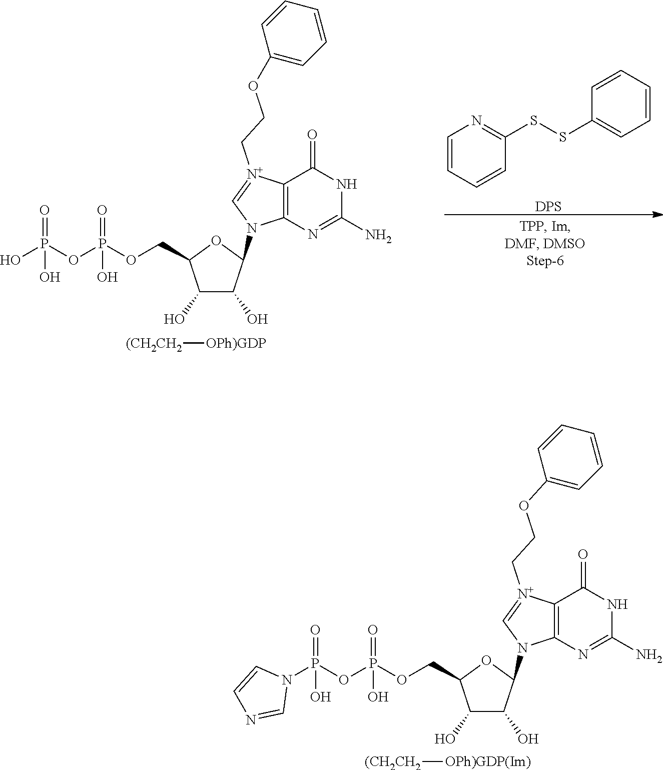

To the solution of (CH₂CH₂—OPh)GDP (150 mg, 0.26 mmol, 1 eq), TPP (423 mg, 1.3 mmol, 5 eq), imidazole (177 mg, 2.6 mmol, 10 eq) in the mixture of DMF:DMSO (2 mL:0.3 mL) was added DPS (286 mg, 1.3 mmol, 5 eq) at 0° C. then allowed to stir at room temperature for 2 h. The reaction mixture was cooled to 0° C. and chilled sodium perchlorate in acetone solution was then added. The formed precipitate was collected by centrifuge and washed thoroughly with acetone then dried. The product (CH₂CH₂—OPh)GDP-Im was isolated; LCMS m/z: 612.0 (M-2H)⁺.

### Synthesis of 2-amino-9-((2R,3R,4S,5R)-5-((((((((((2R,3R,4R,5R)-3-(((((2R,3R,4R,5R)-5-(2-benzamido-6-oxo-1,6-dihydro-9H-purin-9-yl)-3,4-bis(isobutyryloxy)tetrahydrofuran-2-yl)methoxy)(hydroxy)phosphoryl)oxy)-5-(6-benzamido-9H-purin-9-yl)-4-methoxytetrahydrofuran-2-yl)methoxy)(hydroxy)phosphoryl)oxy)(hydroxy)phosphoryl)oxy)(hydroxy)phosphoryl)oxy)methyl)-3,4-dihydroxytetrahydrofuran-2-yl)-6-oxo-7-(2-phenoxyethyl)-6,9-dihydro-1H-purin-7-ium (44)

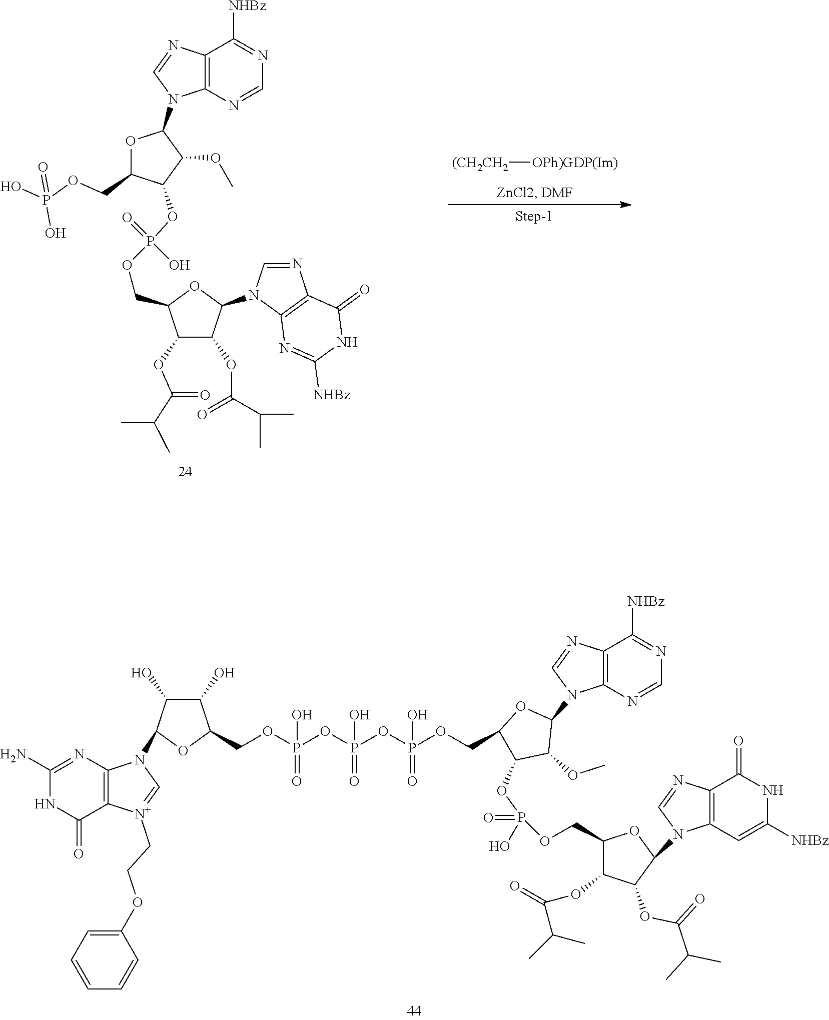

To the stirred solution of compound-24 (; 350 mg, 0.332 mmol, 1 eq) (co-distilled with dry toluene) and (CH₂CH₂—OPh)GDP-Im (305 mg, 0.498 mmol, 1.5 eq) (co-distilled with dry toluene) in dry distilled DMF (10 vol) was added anhydrous ZnCl₂ (0.92 g, 6.64 mmol, 20 eq)(dried under 0.5 mmHg at 60° C. for 2 h) at 0° C. and allowed to warm to room temperature (25° C.) then stirred for 48 h. The reaction mixture was diluted with water (10 mL) at 0° C. and added EDTA solid then the reaction mixture was kept under lyophilization for removal of DMF solvent. After ion exchange DEAE Sephadex chromatography and prep-HPLC purification pure compound-44 was isolated; LCMS m/z: 1598.0 (M-2H)+.

### Step-2: Synthesis of 2-amino-9-((2R,3R,4S,5R)-5-((((((((((2R,3R,4R,5R)-3-(((((2R,3S,4R,5R)-5-(2-amino-6-oxo-1,6-dihydro-9H-purin-9-yl)-3,4-dihydroxytetrahydrofuran-2-yl)methoxy)(hydroxy)phosphoryl) oxy)-5-(6-amino-9H-purin-9-yl)-4-methoxytetrahydrofuran-2-yl)methoxy)(hydroxy)phosphoryl)oxy) (hydroxy)phosphoryl)oxy)(hydroxy)phosphoryl)oxy)methyl)-3,4-dihydroxytetrahydrofuran-2-yl)-6-oxo-7-(2-phenoxyethyl)-6,9-dihydro-1H-purin-7-ium (Compd 2)

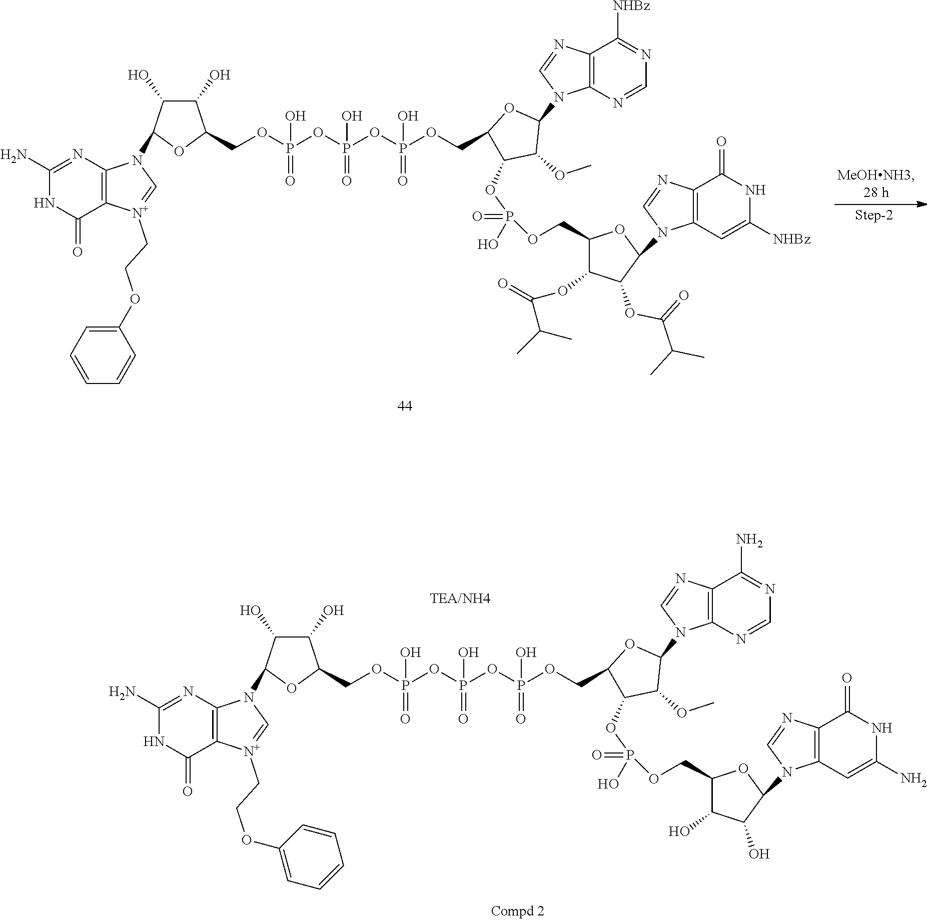

7M MeOH·NH₃ (5 vol*2 times) was added to compound-44 (48 mg, 0.03 mmol, 1 eq) in 25 mL RBF via syringe at 20° C. and stirred for 36 h at same temperature. Reaction progress was monitored by LCMS and after complete consumption of compound-44 proceeded for work-up. After evaporation of volatiles and trituration with diethyl ether, the obtained crude 39 mg/41% was purified by DEAE Sephadex chromatography eluting with 1M ABC/H₂O (After confirming the stability). The obtained semi-pure Compd 2 was further purified by prep-HPLC using 1M aq. ABC and mixture of 10% ACN:30% MeOH:60% water to afford pure Compd 2. LCMS and HPLC indicated 98% purity with desired m/z: 1252.8 (M)+.

### Example 3: Synthesis of CAP 3 (Comparator Example)

### Step-1: Synthesis of N-(9-((2R,3R,4R,5R)-5-((bis(4-methoxyphenyl)(phenyl)methoxy)methyl)-3-methoxy-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-9H-purin-6-yl)benzamide (Int-6A)

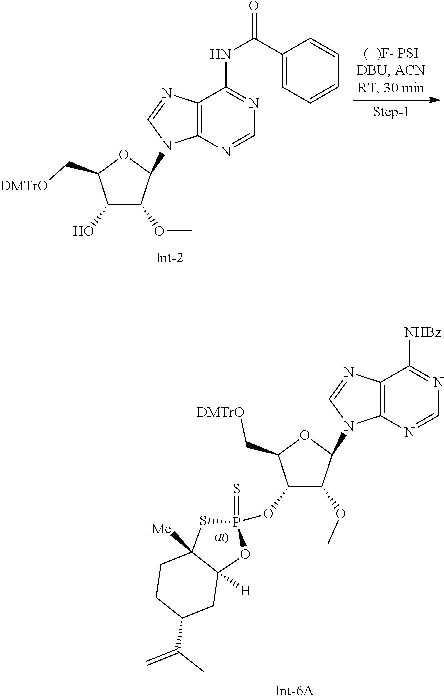

To the stirred solution of Int-2 (Ref: CleanCAP Reagent; 10 g, 14.5 mmol, 1 eq) and (+)-F-PSI reagent (7.1 g, 15.95 mmol, 1.1 eq) in dry distilled ACN (100 mL) at 0° C. was added a solution of DBU (1.4 g, 21.75 mmol, 1.5 eq) in acetonitrile (15 mL) and continued the stirring for 45 min. After complete consumption of (+)-PSI monitored by TLC, the reaction mixture was distilled to reduce acetonitrile volume up to 20 mL. The mixture was purified by silica gel (100-200 mesh) column chromatography eluting with 30% EtOAc in Hexane to afford compound-Int-6A. TLC system:EtOAc/Hexane (30:70), R_{f} value: ^{˜}0.7; LCMS m/z: 933.9 (M+H)⁺.

### Step-2: Synthesis of (2R,3R,4R,5R)-2-(2-benzamido-6-oxo-1,6-dihydro-9H-purin-9-yl)-5-((((R)-(((2R,3R,4R,5R)-5-(6-benzamido-9H-purin-9-yl)-2-((bis(4-methoxyphenyl)(phenyl)methoxy)methyl)-4-methoxytetrahydrofuran-3-yl)oxy)(hydroxy)phosphorothioyl)oxy)methyl)tetrahydrofuran-3,4-diyl bis(2-methylpropanoate) (45)

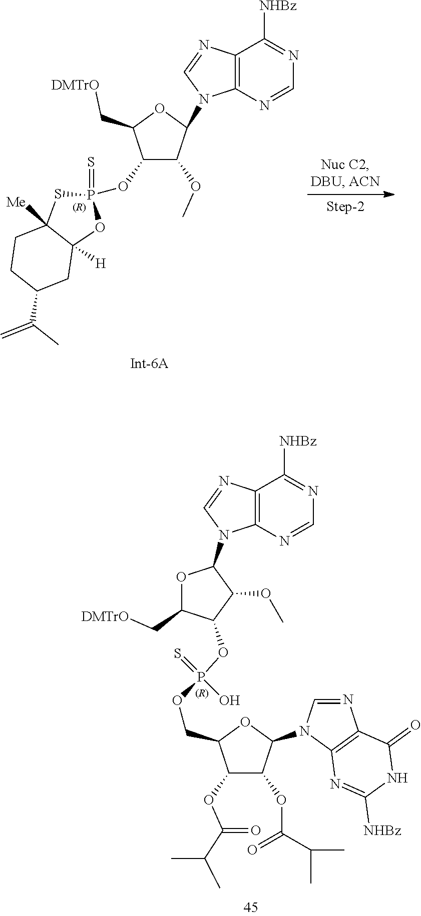

To the stirred solution of Int-6A (10 g, 10.71 mmol, 1 eq) (co-distilled with dry ACN) and Nuc-C2 (7.3 g, 13.92 mmol, 1.3 eq) (co-distilled with dry ACN) in dry distilled ACN (70 mL) was added a solution of dry distilled DBU (2 mL, 16.06 mmol, 1.5 eq) in acetonitrile (15 mL) at 0° C. by drop wise and continued the stirring for 1 h. After complete consumption of starting material by TLC, the reaction mixture was distilled to reduce acetonitrile volume up to 10 mL. The obtained crude solution was purified by C18/Liquid injection reverse phase column chromatography eluting with 1M aq ammonium bicarbonate (ABC) and 40% ACN then the collected pure fraction was lyophilized to afford compound-45. LCMS m/z: 1291.4 (M-2H)⁺.

### Step-3: Synthesis of (2R,3R,4R,5R)-2-(2-benzamido-6-oxo-1,6-dihydro-9H-purin-9-yl)-5-((((R)-(((2R,3R,4R,5R)-5-(6-benzamido-9H-purin-9-yl)-2-(hydroxymethyl)-4-methoxytetrahydrofuran-3-yl)oxy)(hydroxy)phosphorothioyl)oxy)methyl)tetrahydrofuran-3,4-diyl bis(2-methylpropanoate) (46)

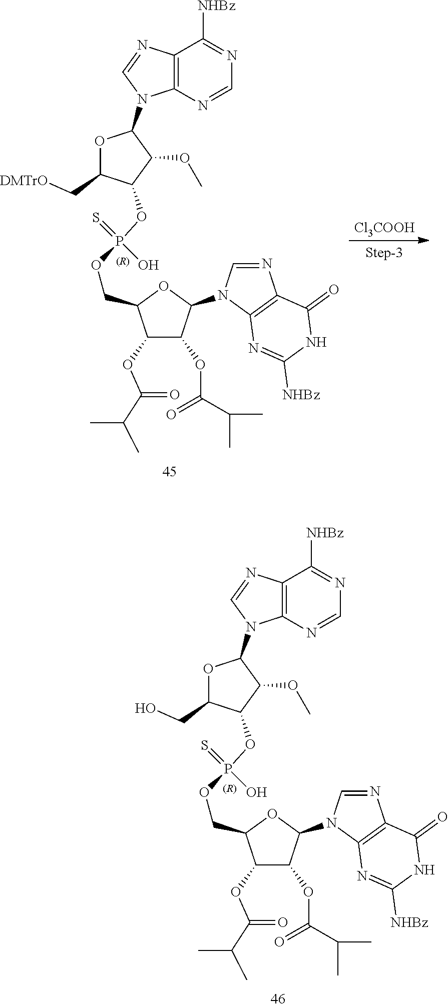

To the stirred solution of compound-45 (4.1 g, 3.17 mmol, 1 eq) in mixture of ACN:DCM (20 mL, 1:1 ratio) was added trichloroacetic acid (1.03 g, 6.34 mmol, 2 eq) at 0° C. the stirred at room temperature for 1 h. Reaction progress was monitored by LCMS. After completion of starting material, the reaction mixture was distillated off under reduced pressure. The obtained semi-pure solid material was purified by C18 reverse phase prep-HPLC using 1M ammonium bicarbonate buffer solution and 40% of ACN. Collected pure fraction was lyophilized to afford compound-46. LCMS m/z: 989.1 (M−H)⁺.

### Step-4: Synthesis of (2R,3R,4R,5R)-2-(2-benzamido-6-oxo-1,6-dihydro-9H-purin-9-yl)-5-((((R)-(((2R,3R,4R,5R)-5-(6-benzamido-9H-purin-9-yl)-4-methoxy-2-((phosphonooxy)methyl)tetrahydrofuran-3-yl)oxy)(hydroxy)phosphorothioyl)oxy)methyl) tetrahydrofuran-3,4-diyl bis(2-methylpropanoate)) (47)

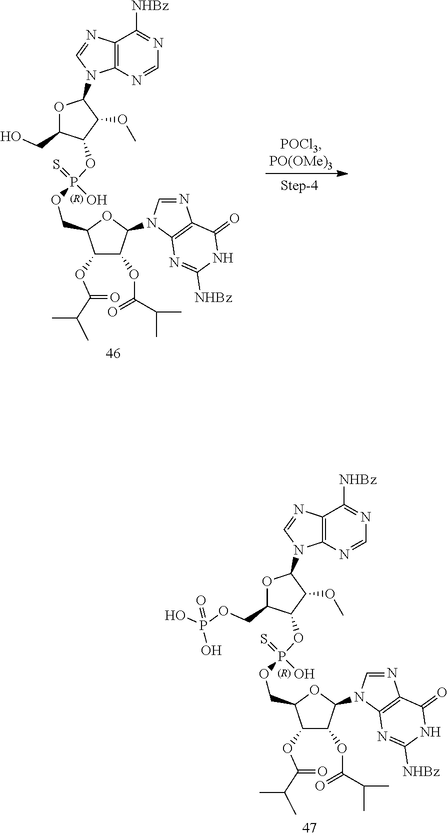

To the stirred solution of compound-46 (1.8 g, 1.81 mmol, 1 eq) in distilled Trimethyl phosphate (1.7 mL, 14.5 mmol, 8 eq) the clear solution was observed after addition of distilled phosphoryl chloride (1.35 mL, 14.5 mmol, 8 eq) at 0° C. for 3 h. Reaction progress was monitored by LCMS, after complete consumption of starting material the reaction mixture was quenched with 1M Triethylammonium bicarbonate (TEAB) then lyophilized. The obtained semi-pure product was purified by C18 reverse phase prep-HPLC eluting with 1M aq. ammonium bicarbonate and 15-20% ACN. The collected pure fraction was lyophilized to afford compound-47. LCMS m/z: 1070.9 (M+H)⁺.

### Step-5: Synthesis of 2-amino-9-((2R,3R,4S,5R)-5-((((((((((2R,3R,4R,5R)-3-(((R)-(((2R,3R,4R,5R)-5-(2-benzamido-6-oxo-1,6-dihydro-9H-purin-9-yl)-3,4-bis(isobutyryloxy)tetrahydrofuran-2-yl)methoxy) (hydroxy)phosphorothioyl)oxy)-5-(6-benzamido-9H-purin-9-yl)-4-methoxytetrahydrofuran-2-yl) methoxy)(hydroxy)phosphoryl)oxy)(hydroxy)phosphoryl)oxy)(hydroxy)phosphoryl)oxy)methyl)-3,4-dihydroxytetrahydrofuran-2-yl)-7-methyl-6-oxo-6,9-dihydro-1H-purin-7-ium (48)

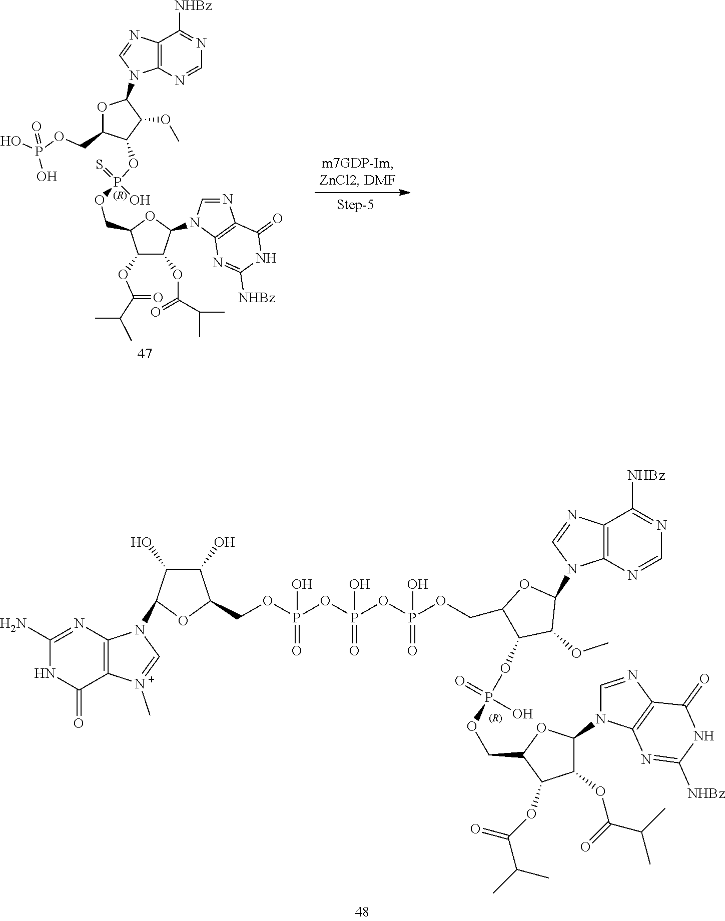

To the stirred solution of compound-47 (400 mg, 0.37 mmol, 1 eq) (co-distilled with dry toluene) and m7GDP-Im (308 mg, 0.67 mmol, 1.8 eq) (co-distilled with dry toluene) in dry distilled DMF (10 vol) was added anhydrous ZnCl₂ (1.25 g, 9.25 mmol, 25 eq) (dried under 0.5 mmHg at 60° C. for 4 h) at 0° C. and allowed to warm to room temperature (25° C.) then stirred for 16 h. After quench the reaction mixture with cooled water then added EDTA solid and the obtained clear solution was lyophilized for removal DMF. After two attempts of DEAE Sephadex column purification eluting with 1M TEAB/ACN and lyophilization, 180 mg of compound 48 was obtained as TEA salt. Compound 48 was isolated after purification by C18 reverse phase column chromatography. LCMS m/z: 1507.9 (M-2H)⁺.

### Step-6: Synthesis of 22-amino-9-((2R,3R,4S,5R)-5-((((((((((2R,3R,4R,5R)-3-(((R)-(((2R,3S,4R,5R)-5-(2-amino-6-oxo-1,6-dihydro-9H-purin-9-yl)-3,4-dihydroxytetrahydrofuran-2-yl)methoxy)(hydroxy) phosphorothioyl)oxy)-5-(6-amino-9H-purin-9-yl)-4-methoxytetrahydrofuran-2-yl)methoxy)(hydroxy) phosphoryl)oxy)(hydroxy)phosphoryl)oxy) (hydroxy)phosphoryl)oxy)methyl)-3,4-dihydroxytetrahydrofuran-2-yl)-7-methyl-6-oxo-6,9-dihydro-1H-purin-7-ium (Cap 3)

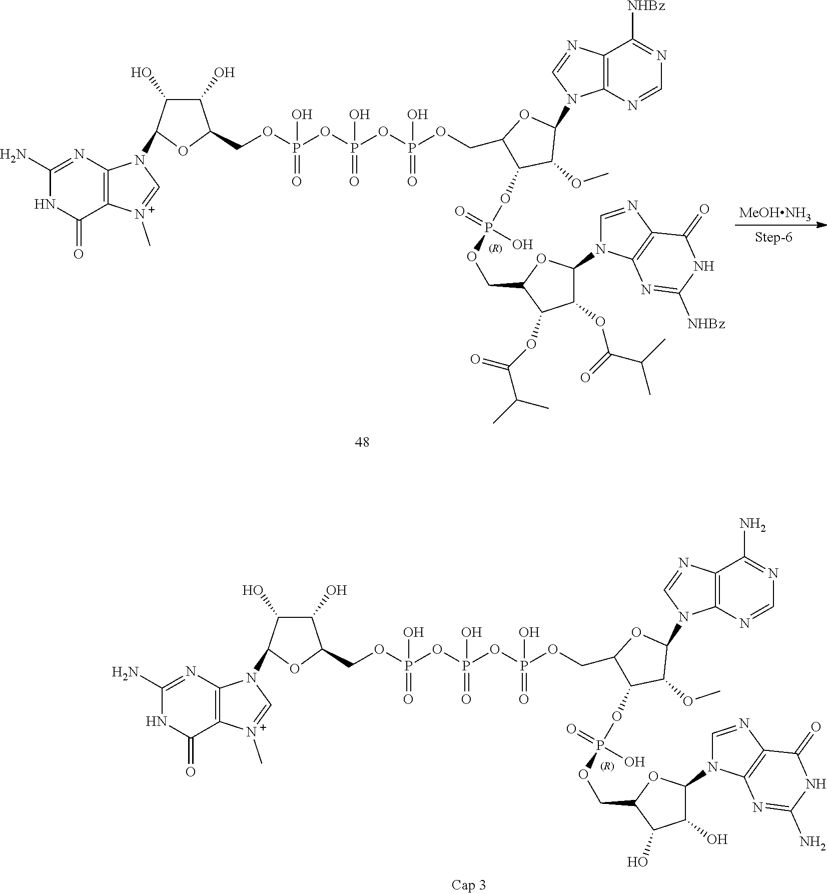

7M MeOH·NH₃ (5 vol*2 times) was added to compound-48 (41 mg, 0.027 mmol, 1 eq) in 25 mL RBF via syringe at 20° C. and stirred for 24 h at same temperature. Reaction progress was monitored by LCMS and after complete consumption of compound-48 proceeded for work-up. After evaporation of volatiles 41.5 mg of crude compound was obtained which was purified by ion exchange DEAE Sephadex and prep-HPLC to afford Cap 3. LCMS & HPLC indicated 93% purity with desired m/z: 1162.1 (M)⁺.

### Example 4: Synthesis of Compound 3 (Compd 3)

### Step-1: Synthesis of (2R,3R,4R,5R)-2-(2-benzamido-6-oxo-1,6-dihydro-9H-purin-9-yl)-5-((((S)-(((2R,3R,4R,5R)-5-(6-benzamido-9H-purin-9-yl)-2-(hydroxymethyl)-4-methoxytetrahydrofuran-3-yl)oxy)(hydroxy)phosphorothioyl)oxy)methyl)tetrahydrofuran-3,4-diyl bis(2-methylpropanoate) (41)

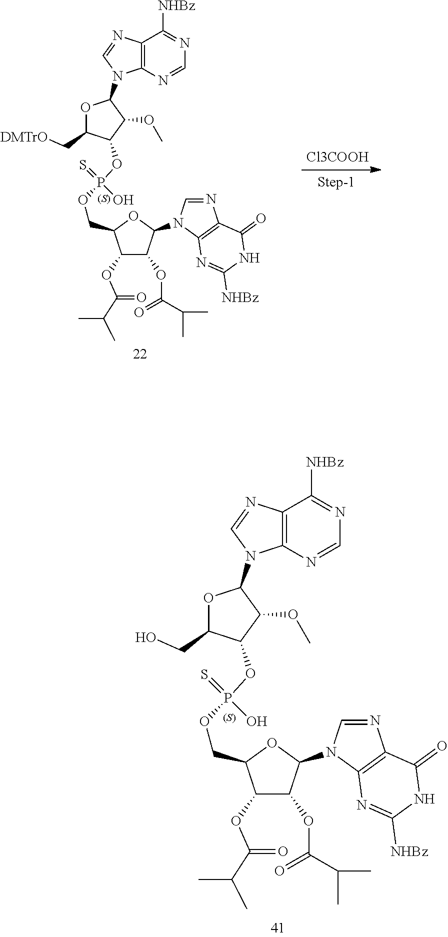

To the stirred solution of compound-22 (Ref: CleanCAP Reagent; 3.3 g, 2.55 mmol, 1 eq) in mixture of ACN:DCM (20 mL, 1:1 ratio) was added trichloroacetic acid (0.83 g, 5.10 mmol, 2 eq) at 0° C. the stirred at room temperature for 1 h. Reaction progress was monitored by LCMS. After complete consumption of starting material the reaction mixture was distilled under reduced pressure. The obtained crude solid material was purified by C18 reverse phase Prep-HPLC using 1M ammonium bicarbonate buffer solution and 40% of ACN. Collected pure fraction was lyophilized to afford compound-41. LCMS m/z: 991.0 (M+H)⁺.

### Step-2: Synthesis of (2R,3R,4R,5R)-2-(2-benzamido-6-oxo-1,6-dihydro-9H-purin-9-yl)-5-((((S)-(((2R,3R,4R,5R)-5-(6-benzamido-9H-purin-9-yl)-4-methoxy-2-((phosphonooxy)methyl)tetrahydrofuran-3-yl)oxy)(hydroxy)phosphorothioyl)oxy) methyl)tetrahydrofuran-3,4-diyl bis(2-methylpropanoate)) (42)

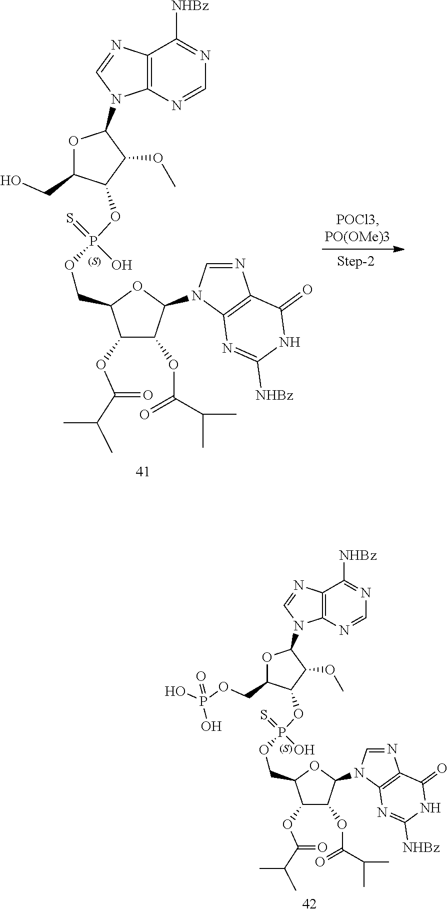

To the stirred solution of compound-41 (1.6 g, 1.61 mmol, 1 eq) in distilled trimethyl phosphate (1.08 mL, 9.28 mmol, 8 eq) the clear solution was observed after addition of distilled phosphoryl chloride (1.5 mL, 16.16 mmol, 10 eq) at 0° C. for 3 h. Reaction progress was monitored by LCMS, after complete consumption of starting material, the reaction mixture was quenched with 1M triethylammonium bicarbonate (TEAB) then purified by C18 reverse phase column chromatography eluting with 1M aq. ammonium bicarbonate and 15-20% ACN. The collected desired fraction was lyophilized to afford compound-42. LCMS m/z: 1071.0 (M+H)⁺.

### Step-1: Synthesis of 2-amino-9-((2R,3R,4S,5R)-5-((((((((((2R,3R,4R,5R)-3-(((S)-(((2R,3R,4R,5R)-5-(2-benzamido-6-oxo-1,6-dihydro-9H-purin-9-yl)-3,4-bis(isobutyryloxy)tetrahydrofuran-2-yl)methoxy)(hydroxy)phosphorothioyl)oxy)-5-(6-benzamido-9H-purin-9-yl)-4-methoxytetrahydrofuran-2-yl)methoxy)(hydroxy)phosphoryl)oxy) (hydroxy)phosphoryl)oxy)(hydroxy)phosphoryl)oxy)methyl)-3,4-dihydroxytetrahydrofuran-2-yl)-7-ethyl-6-oxo-6,9-dihydro-1H-purin-7-ium (49)

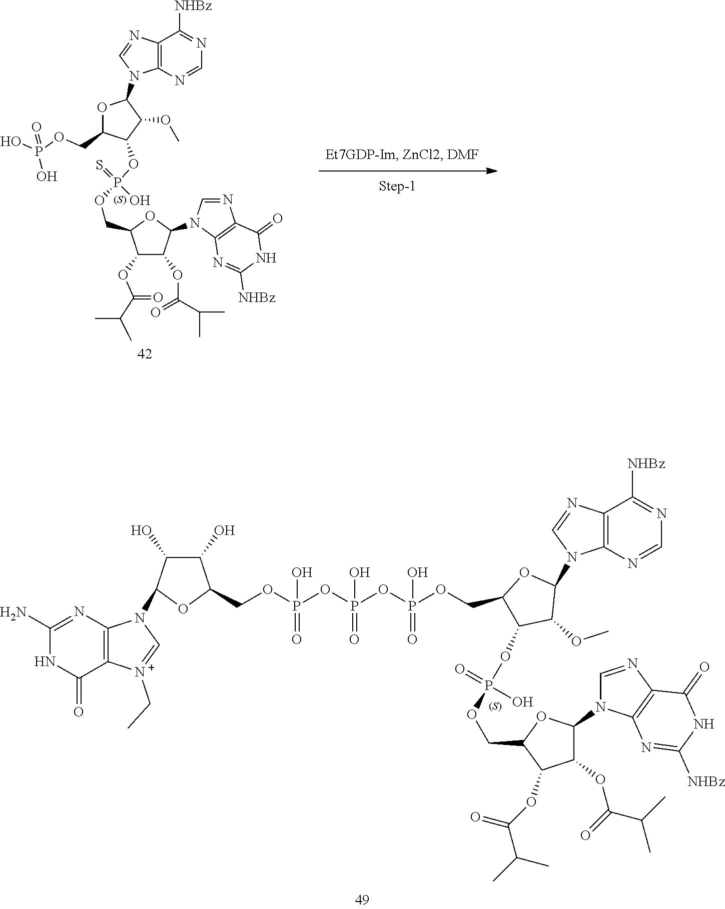

To the stirred solution of compound-42 (390 mg, 0.364 mmol, 1 eq) (co-distilled with dry toluene) and Et7GDP-Im (342 mg, 0.656 mmol, 1.8 eq) (co-distilled with dry toluene) in dry distilled DMF (10 vol) was added anhydrous ZnCl₂ (1.26 g, 9.1 mmol, 25 eq) (dried under 0.5 mmHg at 60° C. for 4 h) at 0° C. and allowed to warm to room temperature (25° C.) then stirred for 48 h.

Stage-1 (Work-up): The reaction mixture was diluted with cold water (10 mL) and EDTA solid then the obtained clear solution was lyophilized for removal of DMF.

Stage-2 (DEAE Sephadex Purification): The obtained crude compound was purified by eluting through DEAE Sephadex with 50% ACN and 1M aq. TEAB. The collected pure fraction was lyophilized to obtain semi-pure Compound-49.

Stage-3 (C18 Purification): Above obtained semi-pure compound was further purified by C18 reverse phase column chromatography eluting with 35% of ACN and 1M aq. ABC to afford pure compound-49. LCMS indicates 89% of desired m/z.

### Step-2: Synthesis of 2-amino-9-((2R,3R,4S,5R)-5-((((((((((2R,3R,4R,5R)-3-(((S)-(((2R,3S,4R,5R)-5-(2-amino-6-oxo-1,6-dihydro-9H-purin-9-yl)-3,4-dihydroxytetrahydrofuran-2-yl)methoxy)(hydroxy) phosphorothioyl)oxy)-5-(6-amino-9H-purin-9-yl)-4-methoxytetrahydrofuran-2-yl)methoxy) (hydroxy)phosphoryl)oxy)(hydroxy)phosphoryl)oxy) (hydroxy)phosphoryl)oxy)methyl)-3,4-dihydroxytetrahydrofuran-2-yl)-7-ethyl-6-oxo-6,9-dihydro-1H-purin-7-ium (Compd 3)

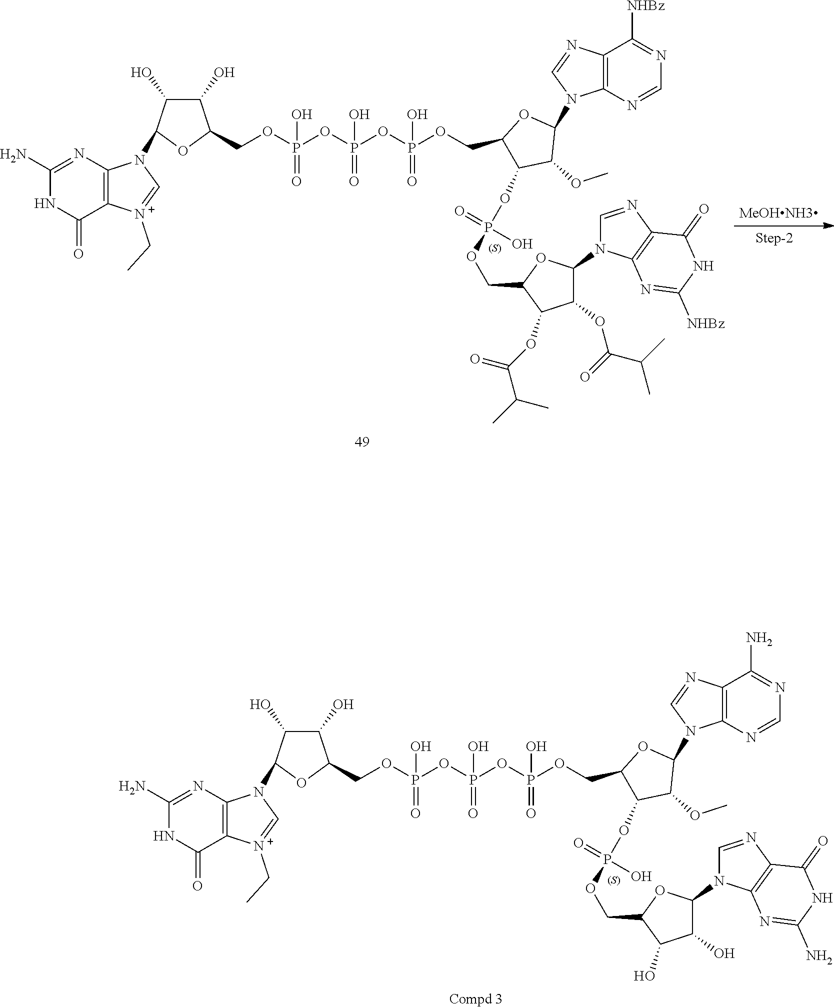

7M MeOH·NH₃ (5 vol*2 times) was added to compound-49 (40 mg, 0.026 mmol, 1 eq) at 20° C. and stirred for 36 h at same temperature. Reaction progress was monitored by LCMS and after complete consumption of compound-49 proceeded for work-up.

Work-up: The reaction mixture was flushed with nitrogen then concentrated under reduced pressure to afford semi-pure compound Compd 3.

DEAE Sephadex Purification: The obtained semi-pure Compd 3 was further purified by eluting through DEAE Sephadex with 2-3% of ACN and 1M aq. TEAB. The collected pure fraction was lyophilized to afford Compd 3 as TEA salt.

Prep-HPLC Purification: The obtained semi-pure Compd 3 was further purified by prep-HPLC using 1M aq. ABC and mixture of 10% ACN:30% MeOH:60% water to afford pure Compd 3. LCMS indicated 98% purity with desired m/z: 1176.0 (M)+.

### Example 5: Synthesis of Compound 4 (Compd 4)

### Step-1: Synthesis of N-(9-((2R,3R,4R,5R)-5-((bis(4-methoxyphenyl)(phenyl)methoxy)methyl)-3-methoxy-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-9H-purin-6-yl)benzamide (Int-6A)

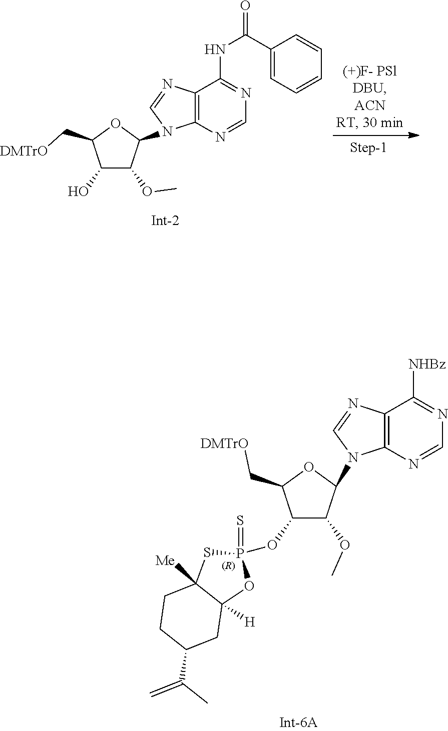

To the stirred solution of Int-2 (Ref: CleanCAP Reagent; 10 g, 14.5 mmol, 1 eq) and (+)-F-PSI reagent (7.1 g, 15.95 mmol, 1.1 eq) in dry distilled ACN (100 mL) at 0° C. was added a solution of DBU (1.4 g, 21.75 mmol, 1.5 eq) in acetonitrile (15 mL) and continued the stirring for 45 min. After complete consumption of (+)-PSI monitored by TLC, the reaction mixture was distilled to reduce acetonitrile volume up to 20 mL. The mixture was purified by silica gel (100-200 mesh) column chromatography eluting with 30% EtOAc in Hexane to afford compound-6A. TLC system:EtOAc/Hexane (30:70), R_{f} value: ^{˜}0.7; LCMS m/z: 933.9 (M+H)⁺.

### Step-2: Synthesis of (2R,3R,4R,5R)-2-(2-benzamido-6-oxo-1,6-dihydro-9H-purin-9-yl)-5-((((R)-(((2R,3R,4R,5R)-5-(6-benzamido-9H-purin-9-yl)-2-((bis(4-methoxyphenyl)(phenyl)methoxy)methyl)-4-methoxytetrahydrofuran-3-yl)oxy)(hydroxy)phosphorothioyl)oxy)methyl)tetrahydrofuran-3,4-diyl bis(2-methylpropanoate) (45)

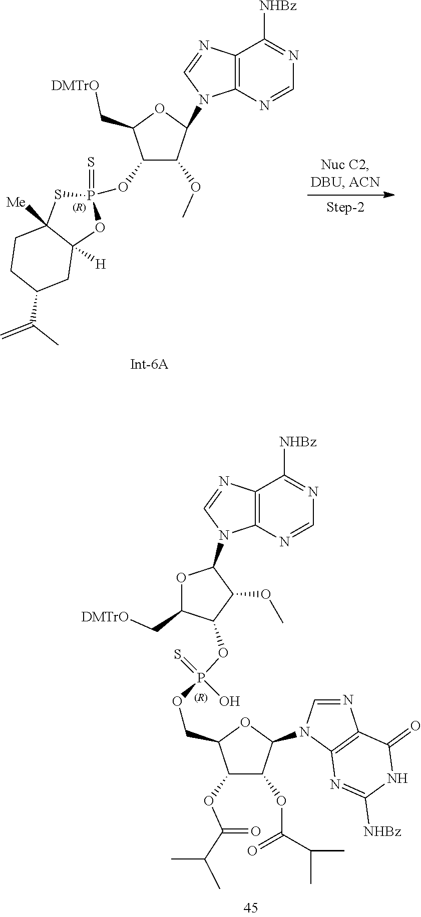

To the stirred solution of Int-6A (10 g, 10.71 mmol, 1 eq) (co-distilled with dry ACN) and Nuc-C2 (7.3 g, 13.92 mmol, 1.3 eq) (co-distilled with dry ACN) in dry distilled ACN (70 mL) was added a solution of dry distilled DBU (2 mL, 16.06 mmol, 1.5 eq) in acetonitrile (15 mL) at 0° C. by drop wise and continued the stirring for 1 h. After complete consumption of starting material by TLC, the reaction mixture was distilled to reduce acetonitrile volume up to 10 mL. The obtained crude solution was purified by C18/Liquid injection reverse phase column chromatography eluting with 1M aq ammonium bicarbonate (ABC) and 40% ACN then the collected pure fraction was lyophilized to afford compound-45. LCMS m/z: 1291.4 (M-2H)⁺.

### Step-3: Synthesis of (2R,3R,4R,5R)-2-(2-benzamido-6-oxo-1,6-dihydro-9H-purin-9-yl)-5-((((R)-(((2R,3R,4R,5R)-5-(6-benzamido-9H-purin-9-yl)-2-(hydroxymethyl)-4-methoxytetrahydrofuran-3-yl)oxy)(hydroxy)phosphorothioyl)oxy)methyl)tetrahydrofuran-3,4-diyl bis(2-methylpropanoate) (46)

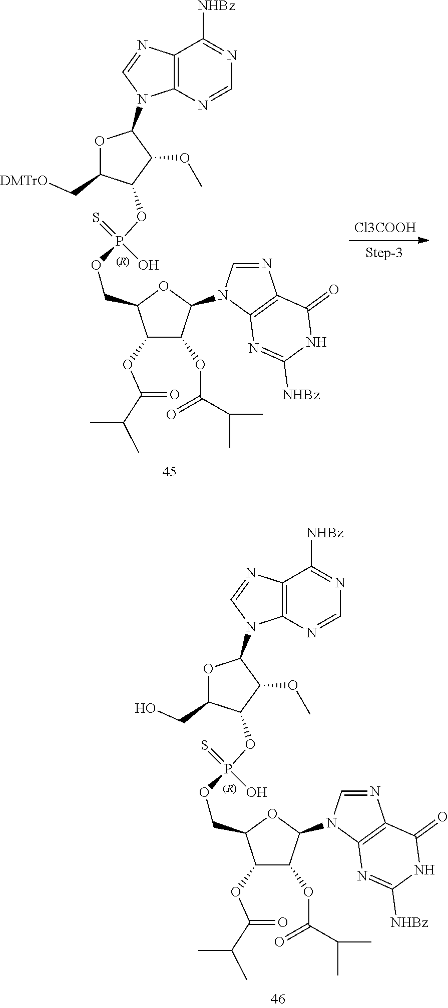

To the stirred solution of compound-45 (4.1 g, 3.17 mmol, 1 eq) in mixture of ACN:DCM (20 mL, 1:1 ratio) was added trichloroacetic acid (1.03 g, 6.34 mmol, 2 eq) at 0° C. the stirred at room temperature for 1 h. Reaction progress was monitored by LCMS. After complete consumption of starting material the reaction mixture was distilled under reduced pressure. The obtained semi-pure solid material was purified by C18 reverse phase Prep-HPLC using 1M ammonium bicarbonate buffer solution and 40% of ACN. Collected pure fraction was lyophilized to afford compound-46. LCMS m/z: 989.1 (M−H)⁺.

### Step-4: Synthesis of (2R,3R,4R,5R)-2-(2-benzamido-6-oxo-1,6-dihydro-9H-purin-9-yl)-5-((((R)-(((2R,3R,4R,5R)-5-(6-benzamido-9H-purin-9-yl)-4-methoxy-2-((phosphonooxy)methyl)tetrahydrofuran-3-yl)oxy)(hydroxy)phosphorothioyl)oxy)methyl) tetrahydrofuran-3,4-diyl bis(2-methylpropanoate)) (47)

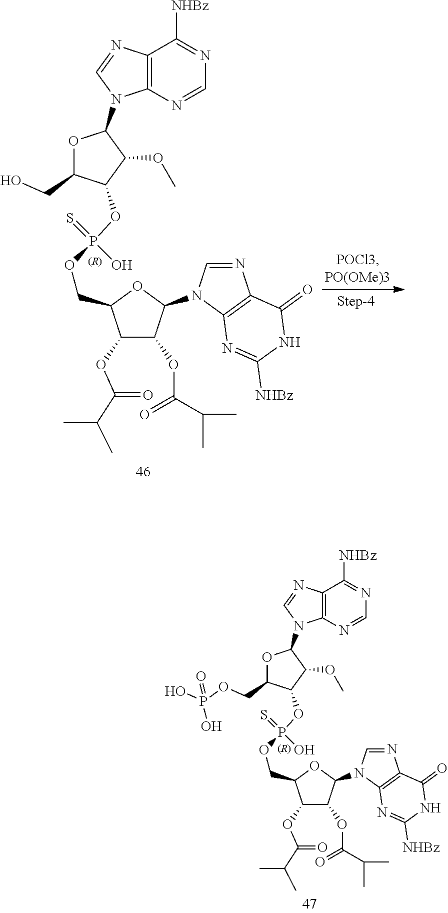

To the stirred solution of compound-46 (1.8 g, 1.81 mmol, 1 eq) in distilled Trimethyl phosphate (1.7 mL, 14.5 mmol, 8 eq) the clear solution was observed after addition of distilled phosphoryl chloride (1.35 mL, 14.5 mmol, 8 eq) at 0° C. for 3 h. Reaction progress was monitored by LCMS, after complete consumption of starting material the reaction mixture was quenched with 1M triethylammonium bicarbonate (TEAB) then lyophilized. The obtained semi-pure was purified by C18 reverse phase prep-HPLC eluting with 1M aq. ammonium bicarbonate and 15-20% ACN. The collected pure fraction was lyophilized to afford compound-47. LCMS m/z: 1070.9 (M+H)⁺

### Step-1: Synthesis of 2-amino-9-((2R,3R,4S,5R)-5-((((((((((2R,3R,4R,5R)-3-(((R)-(((2R,3R,4R,5R)-5-(2-benzamido-6-oxo-1,6-dihydro-9H-purin-9-yl)-3,4-bis(isobutyryloxy)tetrahydrofuran-2-yl)methoxy) (hydroxy)phosphorothioyl)oxy)-5-(6-benzamido-9H-purin-9-yl)-4-methoxytetrahydrofuran-2-yl)methoxy)(hydroxy)phosphoryl)oxy)(hydroxy)phosphoryl)oxy)(hydroxy)phosphoryl)oxy)methyl)-3,4-dihydroxytetrahydrofuran-2-yl)-7-ethyl-6-oxo-6,9-dihydro-1H-purin-7-ium (50)

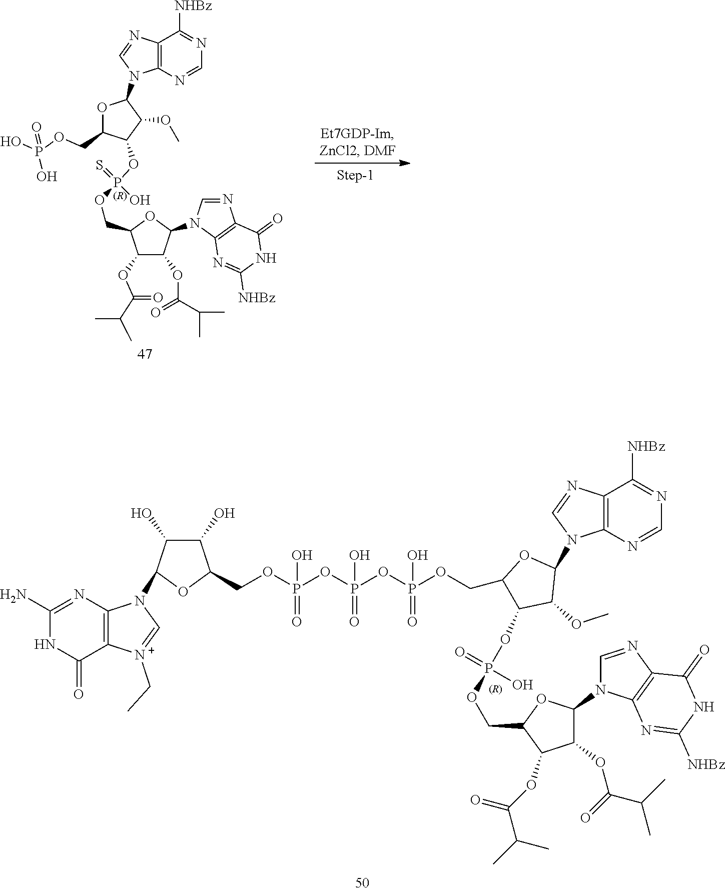

To the stirred solution of compound-47 (400 mg, 0.373 mmol, 1 eq) (co-distilled with dry toluene) and Et7GDP-Im (351 mg, 0.672 mmol, 1.8 eq) (co-distilled with dry toluene) in dry distilled DMF (10 vol) was added anhydrous ZnCl₂ (1.26 g, 9.35 mmol, 25 eq)(dried under 0.5 mmHg at 60° C. for 4 h) at 0° C. and allowed to warm to room temperature (25° C.) then stirred for 48 h.

Stage-1 (Work-up): The reaction mixture was diluted with cold water (10 mL) and EDTA solid then the obtained clear solution was lyophilized for removal of DMF to provide crude material.

Stage-2 (DEAE Sephadex Purification): The obtained crude compound was purified by eluting through DEAE Sephadex with 60% ACN and 1M aq. TEAB. The collected pure fraction was lyophilized to obtain semi-pure Compound-50.

Stage-3 (C18 Purification): Above obtained semi-pure compound was further purified by C18 reverse phase eluting with 55% of ACN and 1M aq. ABC to afford pure compound-50. LCMS indicates 92% of desired m/z.

### Step-2: Synthesis of 2-amino-9-((2R,3R,4S,5R)-5-((((((((((2R,3R,4R,5R)-4-(((((2R,3S,4R,5R)-5-(2-amino-6-oxo-1,6-dihydro-9H-purin-9-yl)-3,4-dihydroxytetrahydrofuran-2-yl)methoxy)(hydroxy)phosphoryl) oxy)-5-(6-amino-9H-purin-9-yl)-3-methoxytetrahydrofuran-2-yl)methoxy)(hydroxy)phosphoryl)oxy) (hydroxy)phosphoryl)oxy)(hydroxy)phosphoryl)oxy)methyl)-3,4-dihydroxytetrahydrofuran-2-yl)-7-methyl-6-oxo-6,9-dihydro-1H-purin-7-ium (Compd 4)

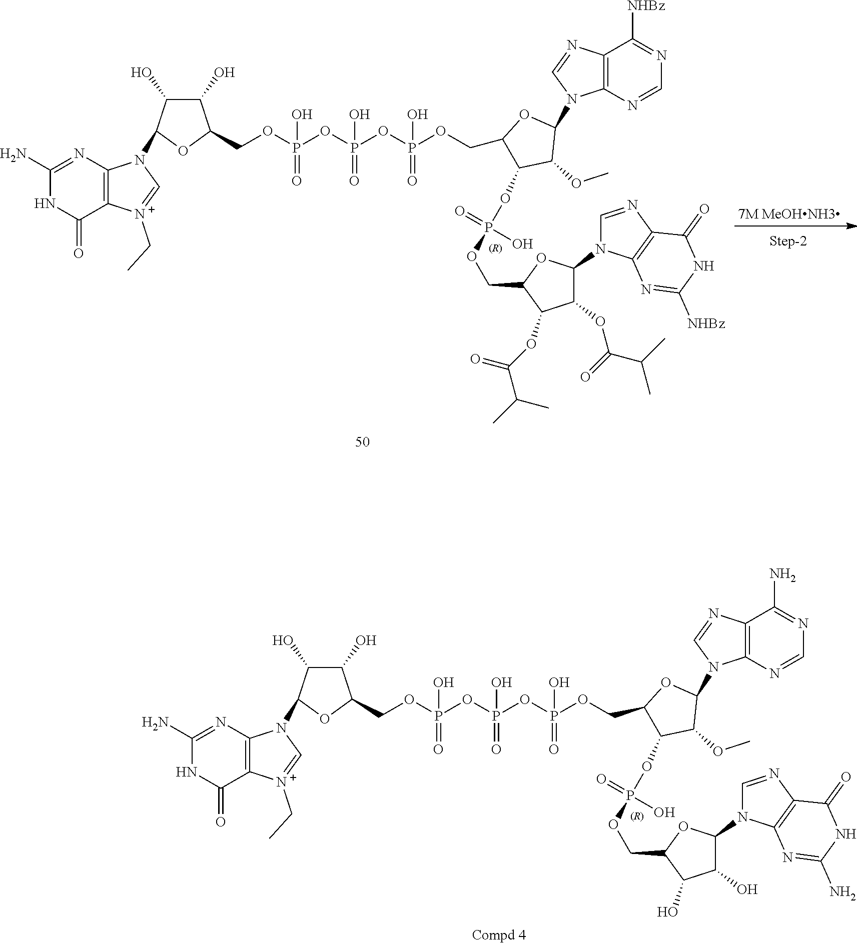

7M MeOH·NH₃ (5 vol*2 times) was added to compound-50 (41 mg, 0.053 mmol, 1 eq) at 20° C. and stirred for 24 h at same temperature. Reaction progress was monitored by LCMS and after complete consumption of compound-50 proceeded for work-up.

Work-up: The reaction mixture was flushed with nitrogen then concentrated under reduced pressure to afford semi-pure Compd 4.

DEAE Sephadex Purification: The obtained semi-pure Compound 4 was further purified by eluting through DEAE Sephadex with 2-3% of ACN and 1M aq. TEAB. The collected pure fraction was lyophilized to afford Compd 4 as TEA salt.

Prep-HPLC Purification: The obtained semi-pure Compd 4 was further purified by prep-HPLC using 1M aq. ABC and mixture of 10% ACN:30% MeOH:60% water to afford pure Compd 4. LCMS indicated 97% purity with desired m/z: 1157.7 (M)⁺.

### Example 6: Relative Yield, Expression and dsRNA of Compound 1

Experiments were conducted as described in the Materials and Methods section, above.

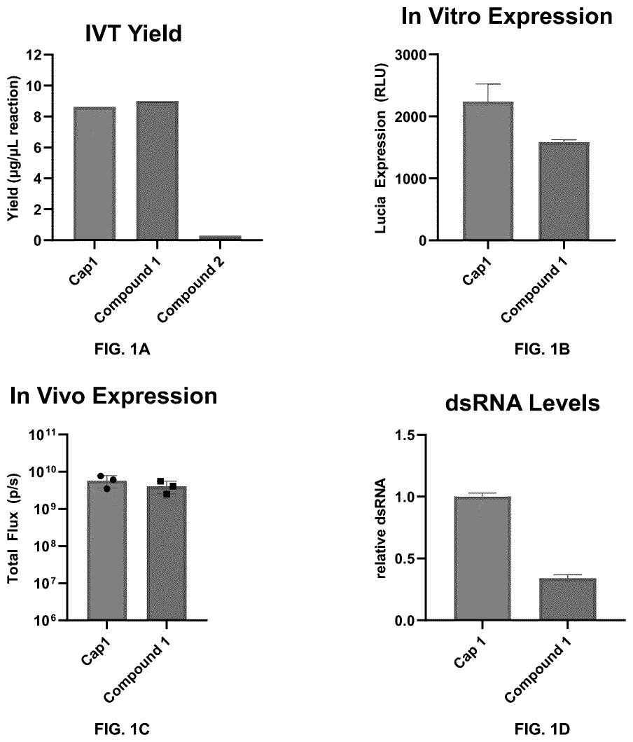

As shown in FIGS. 1A-1D, Cap 1, Cap 2 and Cap 3 all have a standard Me7G structure; Compound 1, Compound 3 and Compound 4 have a novel Et7G structure; and Compound 2 has an aromatic substitution at that position.

Compound 1 performed equivalently to Cap1 for Yield and in vivo expression but produced significantly lower dsRNA levels compared to the Me7G Cap1 structure.

The data show that trinucleotides modified with an ethyl group at the 7G position (Compounds 1, 3 and 4) were successfully incorporated into IVT, but larger modifications at that position (such as an aromatic substitution) were not (cf. Compound 1 and Compound 2).

### Example 7: Relative Yield, Expression and dsRNA of Compound 3

Experiments were conducted as described in the Materials and Methods section, above.

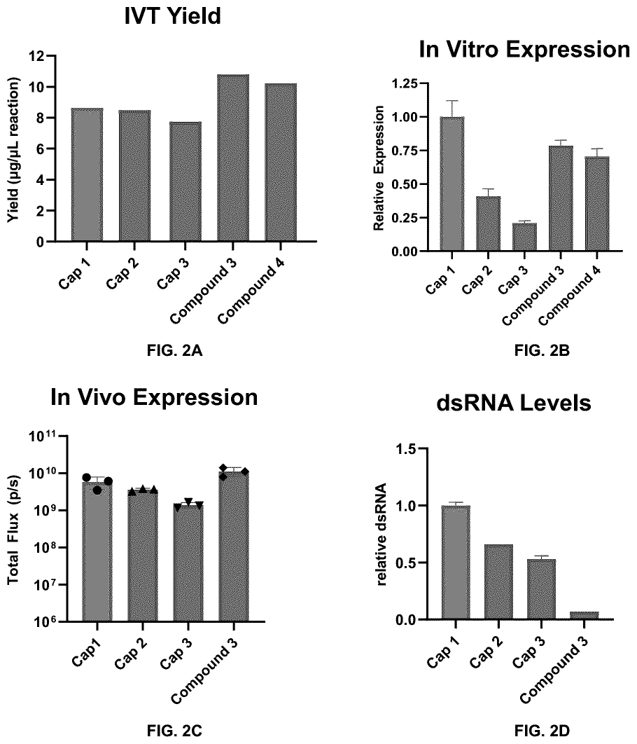

As shown in FIGS. 2A-2D, the Et7G modification on phosphorothioate-modified caps (Compounds 3 & 4) showed a similar IVT yield, but superior expression and lower dsRNA levels, as compared with the comparator Me7G-containing phosphorothioate-modified caps (Caps 2 & 3).

### Example 8: Assessment of Cap Immunogenicity by Multiplexed Screening (In Vitro)

Fresh human Dendritic Cells (differentiated from PBMCs by GM-CSF+IL-4 stimulation) were plated at 5e4 cells per well in 96 well flat TC treated dish. mRNA was prepared in Lipofectamine MessengerMAX transfection reagent according to manufacturer's instructions at 2ng/1 k cells. Briefly, mRNA was diluted to 0.02 μg/μl in Opti-MEM reduced serum medium and mixed 1:1 with MessengerMAX to a concentration of 0.01 μg/μl. Two 1:10 dilutions were performed and 10 or 2.5 μl per well of each dilution was added directly to DCs in resulting doses of 2, 0.5, 0.2, 0.05 and 0.02 ng per 1000 DCs. Immunostimulatory RIG-1 agonist Hairpin RNA (3p-hpRNA, InvivoGen) and double stranded RNA (5′ ppp-dsRNA, InvivoGen) were used as controls and formulated as described above.

Stimulated DCs were incubated for 24 hours at 37° C. with 5% CO₂. Supernatant was collected and immediately frozen at −80° C. It was subsequently thawed and prepared for Luminex xMAP analysis using a Human Inflammation 20-Plex ProcartaPlex Panel kit (ThermoFisher) according to the manufacturer's instructions. Data was acquired on a MAGPIX system (Luminex xMAP instrument) and pg/mL values for each analyte were derived from a standard curve generated with kit components. Values outside the range of the standard curve were reported as mean fluorescence intensity (MFI).

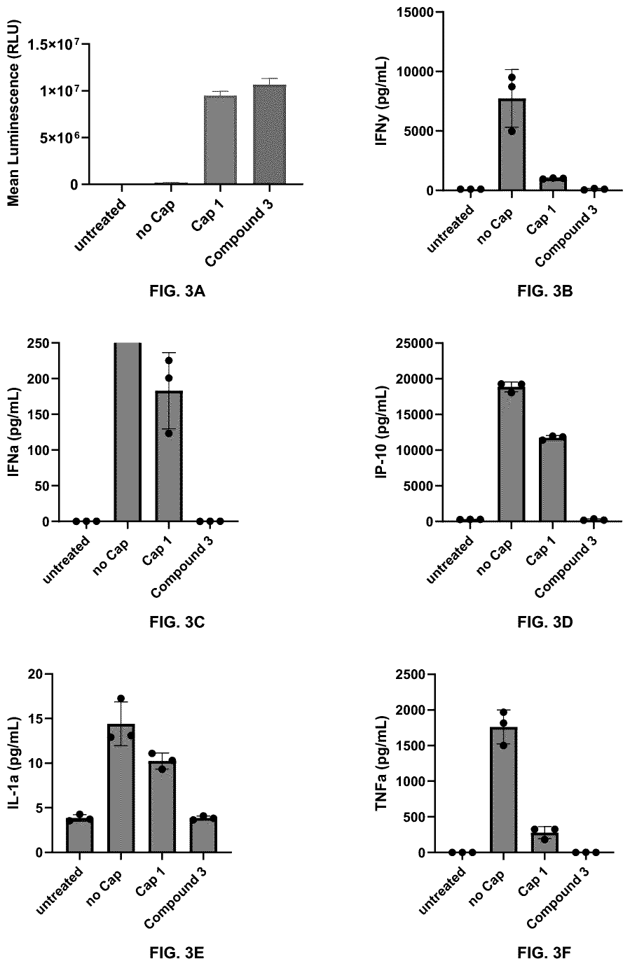

As shown in FIGS. 3A-3F, Compound 3 shows lower immunogenicity than Cap1 across key cytokine markers (including IFNγ, IFNα, IP-10, IL-1α, and TNFα).

1. A compound of Formula (I), or a stereoisomer, tautomer or pharmaceutically acceptable salt thereof: 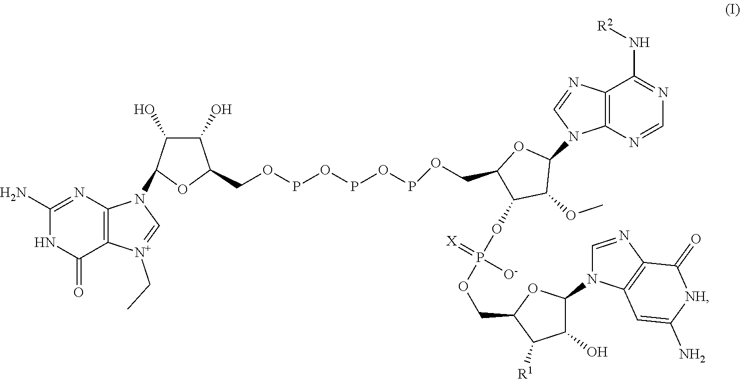
wherein
X is O or S;
R¹ is OH or a polynucleotide, and
R² is H or Me.

2. The compound, stereoisomer, tautomer, or salt of claim 1, wherein X is O.

3. The compound, stereoisomer, tautomer, or salt of claim 1, wherein X is S.

4. The compound, stereoisomer, tautomer, or salt of claim 1, wherein R¹ is OH.

5. The compound, stereoisomer, tautomer, or salt of claim 1, wherein R² is H.

6. The compound, stereoisomer, tautomer, or salt of claim 1, wherein R² is Me.

7. A compound as shown in Table 1, or a stereoisomer, tautomer, or pharmaceutically acceptable salt thereof:
TABLE 1

| Compound ID | Structure |
|---|---|
| Compound 1 (Compd 1) | 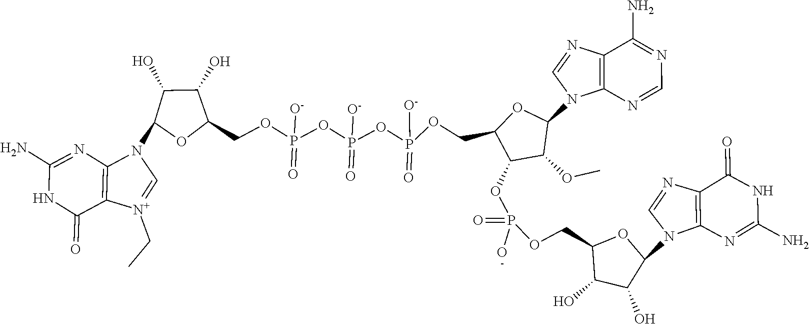 [SMILES: CC[n+]1cn([C@@H]2O[C@H](COP(=O)([O-])OP(=O)([O-])OP(=O)([O-])OC[C@H]3O[C@@H](n4cnc5c(N)ncnc54)[C@H](OC)[C@@H]3OP(=O)(O)OC[C@H]3O[C@@H](n4cnc5c(=O)[nH]c(N)nc54)[C@H](O)[C@@H]3O)[C@@H](O)[C@H]2O)c2nc(N)[nH]c(=O)c21] |
| Compound 2 (Compd 2) | 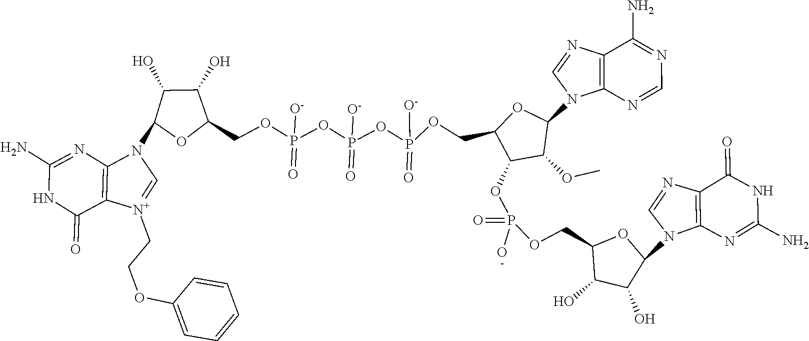 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)([O-])OP(=O)([O-])OP(=O)([O-])OC[C@H]2O[C@@H](n3c[n+](CCOc4ccccc4)c4c(=O)[nH]c(N)nc43)[C@H](O)[C@@H]2O)O[C@H]1n1cnc2c(N)ncnc21] |
| Compound 3 (Compd 3) | 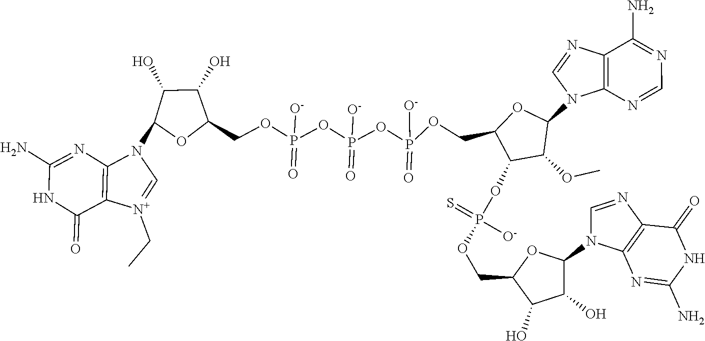 [SMILES: CC[n+]1cn([C@@H]2O[C@H](COP(=O)([O-])OP(=O)([O-])OP(=O)([O-])OC[C@H]3O[C@@H](n4cnc5c(N)ncnc54)[C@H](OC)[C@@H]3O[P@@]([O-])(=S)OC[C@H]3O[C@@H](n4cnc5c(=O)[nH]c(N)nc54)[C@H](O)[C@@H]3O)[C@@H](O)[C@H]2O)c2nc(N)[nH]c(=O)c21] |
| Compound 4 (Compd 4) | 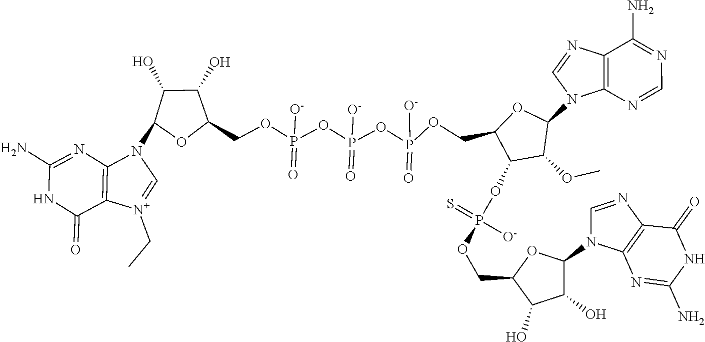 [SMILES: CC[n+]1cn([C@@H]2O[C@H](COP(=O)([O-])OP(=O)([O-])OP(=O)([O-])OC[C@H]3O[C@@H](n4cnc5c(N)ncnc54)[C@H](OC)[C@@H]3O[P@]([O-])(=S)OC[C@H]3O[C@@H](n4cnc5c(=O)[nH]c(N)nc54)[C@H](O)[C@@H]3O)[C@@H](O)[C@H]2O)c2nc(N)[nH]c(=O)c21] |

8. An RNA molecule, wherein the 5′ end of the RNA molecule comprises the compound, stereoisomer, tautomer, or salt of claim 1.

9. The RNA molecule of claim 8, wherein the RNA molecule is mRNA.

10. The RNA molecule of claim 9, wherein the mRNA encodes for one or more immunomodulatory agents (e.g., a checkpoint inhibitor such as an anti-PD-1 antibody, anti-PDL-1 antibody, anti-CTLA4, etc., an immunosuppression antagonist, a pro-inflammatory agent/cytokine), therapeutic proteins, antibodies, or antigens (e.g., a tumor specific antigen).

11. The RNA molecule of claim 8, wherein the RNA molecule has a half-life that is more than that of a corresponding natural RNA molecule in a cellular environment.

12. The RNA molecule of claim 8, having reduced formation of double stranded RNA compared to an RNA molecule with a 5′ end not comprising the compound, stereoisomer, tautomer, or salt of any one of claims 1 to 7.

13. A drug product comprising the RNA molecule of claim 8 and one or more pharmaceutically acceptable excipients.

14. A kit for capping an RNA molecule comprising the compound, stereoisomer, tautomer, or salt of claim 1, and an RNA polymerase.

15. (canceled)

16. A method for making a capped RNA molecule from a polynucleotide template by in vitro transcription, comprising:
(a) combining a plurality of nucleotides, the polynucleotide template and an RNA polymerase to produce a reaction mix;
(b) incubating the reaction mix; and
(c) adding the compound, stereoisomer, tautomer, or salt of claim 1 to the mix in order to produce the capped RNA molecule.

17. The method of claim 16, wherein the RNA molecule is mRNA.

18. The method of claim 16, wherein at least a portion of the plurality of nucleotides are unmodified.

19. The method of claim 16, wherein the at least a portion of the plurality of nucleotides are modified.

20. The method of claim 16, wherein the polynucleotide template is a DNA template.

21. The method of claim 16, wherein the capping efficiency is greater than about 95%, the translation yield is greater than about 75%, or both.

22. (canceled)
